(19) United States
(12) Patent Application Publication
(10) Pub. No.: US 2016/0244486 A1
(43) Pub. Date: Aug. 25, 2016

(54) AB Modulating Peptides

(71) Applicant: ALZHYME PTY LTD, Bentley, Western Australia (AU)

(72) Inventors: Ralph Martins, Bentley, Western Australia (AU); Giuseppe Verdile, Bentley, Western Australia (AU); Kevin Taddei, Bentley, Western Australia (AU); Renae Barr, Bentley, Western Australia (AU)

(73) Assignee: Alzyme Pty Ltd., Bentley, Western Australia (AU)

(21) Appl. No.: 15/030,077

(22) PCT Filed: Oct. 21, 2014
(86) PCT No.: PCT/AU2014/050298

§ 371 Date: Apr. 18, 2016

(30) Foreign Application Priority Data
Oct. 22, 2013 (AU) 2013904077
Apr. 17, 2014 (AU) 2014901419

(51) Int. Cl. C07K 7/08; C07K 7/06; G01N 33/68

(52) U.S. Cl. CPC C07K 7/08; G01N 33/6896; C07K 7/06; G01N 2800/2821; A61K 38/00

(57) ABSTRACT

The present invention relates to an Aβ modulating peptide comprising a peptide selected from the list of peptides comprising: (i) Arg-Lys-Leu-Met-Gln-Pro-Thr-Arg-Asn (SEQ ID NO:1); (ii) Arg-Lys-Leu-Met-Gln-Pro-Thr-Arg-Asn-Arg-Arg-Asn-Pro-Asn-Thr (SEQ ID NO:2); (iii) a peptide according to (i) or (ii) wherein at least one of the amino acids is a D-amino acid; (iv) a functional variant of a peptide according to any one of (i) to (iii); and (v) a peptide consisting of at least 3-5 contiguous amino acid residues of a peptide according to (i), (ii), (iii) or (iv). The present invention also relates to an Aβ modulating peptide comprising SEQ ID NO: 2 wherein all of the amino acids are D-amino acids.

### FIELD OF THE INVENTION

This invention relates to peptides, and pharmaceutical compositions thereof, that are adapted to modulate Aβ. These Aβ modulating peptides can be used to modulate Aβ and have various affects on Aβ that can lead to positive therapeutic outcomes. The present invention also relates to the use of the Aβ modulating peptides as imaging agents for diagnosis and, in addition, to methods of treating AD as well as antibodies to the Aβ modulating peptides.

### BACKGROUND

The amyloid related disease Alzheimer's disease (AD) is a progressive neurodegenerative disorder characterised pathologically by the deposition of amyloid plaques and neurofibrillary tangles, and neuronal degeneration, in the brains of affected individuals. According to the WHO Dementia report 2012, the world-wide incidence for Dementia is estimated to be 115.4 million in 2050.

The major protein component of the amyloid deposits is a small 4 kDa peptide of 39-43 amino acids termed beta amyloid (β-amyloid or Aβ). Aβ is a small protein thought to be central to the pathogenesis of AD. Numerous studies have suggested that Aβ accumulation and deposition may be critical to AD. The initial deposition of Aβ and growth of plaques has been suggested to occur via distinct processes. Aβ may either form higher oligomeric structures or remain in the monomeric form when it is deposited. In vitro studies have found that freshly solubilised monomeric Aβ, at low concentrations, is not toxic to neurons in culture. However, after an aging period of several hours to days Aβ spontaneously aggregates in solution to form fibrillar entities that are highly neurotoxic. This suggests aggregation is a requirement for Aβ toxicity.

There remains a need for new and improved agents for treating and diagnosing amyloid related diseases such as Alzheimer's disease. The present invention seeks to address this need.

### SUMMARY OF THE INVENTION

In one aspect, the present invention provides an Aβ modulating peptide comprising a peptide selected from the list of peptides comprising:

(i)
(SEQ ID NO: 1)
Arg-Lys-Leu-Met-Gln-Pro-Thr-Arg-Asn;
(ii)
(SEQ ID NO: 2)
Arg-Lys-Leu-Met-Gln-Pro-Thr-Arg-Asn-Arg-Arg-
Asn-Pro-Asn-Thr;

(iii) a peptide according to (i) or (ii) wherein at least one of the amino acids is a D-amino acid;

(iv) a functional variant of a peptide according to any one of (i) to (iii); and

(v) a peptide consisting of at least 3-5 contiguous amino acid residues of a peptide according to (i), (ii), (iii) or (iv).

Preferably, the Aβ modulating peptide comprises at least 1-5, 6-9 or 10-15 D-amino acids. In one embodiment the Aβ modulating peptide is: Arg-Lys-Leu-Met-Gln-Pro-Thr-Arg-Asn-Arg-Arg-Asn-Pro-Asn-Thr (SEQ ID NO:2); wherein all of the amino acids are D-amino acids.

In another aspect, the present invention provides a pharmaceutical composition comprising an Aβ modulating peptide as herein described and a pharmaceutically acceptable carrier.

According to another aspect, the present invention provides a method for modulating aggregation or neurotoxicity of Aβ or peripheral clearance of Aβ comprising the step of contacting Aβ with an Aβ modulating peptide according to the present invention.

A further aspect of the present invention provides a method for detecting the presence or absence of Aβ comprising the step of contacting a sample with an Aβ modulating peptide according to the present invention and detecting the formation of a complex between the Aβ and the Aβ modulating peptide. Preferably, the detection enables the diagnosis of amyloidosis in a subject.

A still further aspect of the present invention provides a method for treating a subject with amyloidosis comprising the step of administering to said subject an effective amount of an Aβ modulating peptide according to the present invention. Preferably, the amyloidosis is Alzheimer's disease.

According to another aspect of the present invention there is provided a polynucleotide encoding a peptide according to the present invention.

### BRIEF DESCRIPTION OF THE DRAWINGS

FIG. 1A is a graph demonstrating the effect of Aβ modulating peptide RI-ANA1 on aggregation of monomeric Aβ42 over time (Aβ/Aβ42=monomeric Aβ42; Scr 9 mer=scrambled ANA5; and 15 mer S.A.=RI-ANA1);

FIG. 1B is a graph demonstrating the effect of Aβ modulating peptides RI-ANA1 on aggregation of monomeric Aβ42 where samples are “spiked' with the peptides during the time course assay (Aβ/Aβ42=monomeric Aβ42; Scr 9 mer=scrambled ANA5; 15 mer S.A.=RI-ANA1 and 9 mer=ANA5),

FIG. 1C is a graph demonstrating the effect of the Aβ modulating peptides RI-ANA1 on aggregation of Aβ42 under conditions favouring oligomerisation based on a ThT endpoint assay (Aβ/Aβ42=oligomeric Aβ42; Scr 9 mer=scrambled ANA5; 9 mer=ANA5; 9 mer S.A.=RI-ANA5; 15 mer S.A.=RI-ANA1 and 15 mer=ANA1);

FIG. 2A is a graph demonstrating the effect of the Aβ modulating peptide RI-ANA1 on neurotoxicity and an image of a gel analysis of the Aβ aggregation in the soluble fraction as represented by the “smear' (Aβ/Aβ42=monomeric Aβ42; Scr 9 mer=scrambled ANA5; 9 mer=ANA5; 9 mer S.A.=RI-ANA5; 15 mer S.A.=RI-ANA1 and 15 mer=ANA1);

FIG. 2B is an image of a gel analysis of the peptides resulting from a combination of RI-ANA1 and Aβ42, comparing the Aβ species present in the soluble versus insoluble fraction (Aβ/Aβ42=monomeric Aβ42; Scr 9 mer=scrambled ANA5; 9 mer ANA5, 9 mer S.A.=RI-ANA5; 15 mer S.A.=RI-ANA1 and 15 mer=ANA1);

FIG. 3A is an image of a gel analysis of the peptide mixtures resulting from the combination of Aβ modulating peptide RI-ANA1 and oligomeric Aβ42 (oAβ42=oligomeric Aβ42, Scr 9 mer=scrambled ANA5; 15 mer S.A.=RI-ANA1);

FIG. 4A is a graph showing clearance of Al342 from plasma of 12-month old male human APOE4 knock-in (targeted replacement mice) following tail vein injection of 20 μg/50 μl Aβ42 determined by western blot quantification. Values are mean±SEM. *p=0.013 vs Aβ42+saline 2.5 min; #p=0.023 vs Aβ42+0.5 mg RI-ANA1 2.5 min; §p=0013 vs Aβ42+saline 20 min (Abeta42=monomeric Aβ42);

FIG. 4B is a graph showing levels of Aβ42 in liver of 12-month old male human APOE4 knock-in (targeted replacement mice) following tail vein injection of 20 μg/50 μl Aβ42 determined by western blot quantification. Values are mean±SEM;

FIGS. 5A and 5B are a graph and an image of BN PAGE/Western blot showing the effect of several peptides, including ANA1 and 15M S.A. (RI-ANA1) on Aβ42 aggregation. The broken line in FIG. 5B indicates that data has been spliced to ease viewing but all data is from the same experiment;

FIG. 5C is a graph showing the effect of the candidate peptides including 15M S.A. (RI-ANA1) on the toxicity of oligomeric Aβ42 in M17 neuroblastoma cells;

FIG. 6A is an image of SDS PAGE/Western blot showing the effect of peptide 15M SA. (RI-ANA1) on the formation of soluble and insoluble Aβ42. The asterisks indicate samples with a reduction in soluble Aβ42 aggregates;

FIG. 6B are images taken using atomic force microscopy of combined soluble/insoluble Aβ42 aggregates formed in the presence or absence of the 15M SA. (RI-ANA1) peptide under conditions favouring oligomerisation;

FIG. 7A(i) and (ii) are a sensor gram and a bar chart of maximal response illustrating the binding of Aβ42 oligomers to immobilised 15M S.A. (RI-ANA1) peptide on a CM5 sensorchip;

FIG. 7B(i) and (ii) are a sensor gram and a bar chart of maximal response illustrating the (reduced) binding of Aβ42 oligomers to immobilised 15M S.A. (RI-ANA1) peptide on a CM5 sensorchip in the presence of free 15M S.A (RI-ANA1),

FIG. 7C is an image and table further demonstrating the binding of 15M S.A. (RI-ANA1) to Aβ42 using coimmunoprecipitation;

FIG. 8 is a graph demonstrating the binding of 15M S.A. (RI-ANA1) to monomeric (triangle), oligomeric (diamond) and fibrillar (square) Aβ42 immobilised on individual cells of a CM5 sensorchip;

FIG. 9 depicts images of ex-vivo staining of serial sagittal-sections from the subiculum of 8 month old AD model mice (5× FAD) or age-matched non-transgenic controls (Non-Tg) (10mM thickness, 10× magnification, scale bar=200 mm; inset=40× magnification) where amyloid deposits were detected by thioflavin S staining in 5× FAD mice (A). TMR-labelled 15M SA. (RI-ANA1) peptide in 5× FAD mice (B), TMR-labelled 15M S.A. (RI-ANA1) in Non-Tg control mice (C) and TMR-labelled control peptide (CTL2 S.A.) in 5× FAD mice (D);

FIG. 10A is a graph showing the presence of i.v. (tail vein) administered 15M S.A. (RI-ANA1) in the brains of treated Male Swiss Outbred mice with reference to the concentration of 15M SA (RI-ANA1) in the brain and the plasma;

FIG. 10B is a chart showing the presence of i.v. (tail vein) administered 15M S.A. (RI-ANA1) in the brains of treated Male Swiss Outbred mice with reference to the brain to plasma ratio of 15M S.A. (RI-ANA1); and

FIG. 10C and 10D are graphs showing the intactness of the 15M S.A. (RI-ANA1) in brain (FIG. 10C) and plasma (FIG. 10D).

### DETAILED DESCRIPTION OF THE INVENTION

Aβ Modulating Peptides

In one embodiment, the present invention provides an Aβ modulating peptide comprising a peptide selected from the list of peptides comprising:

(i)
(SEQ ID NO: 1)
Arg-Lys-Leu-Met-Gln-Pro-Thr-Arg-Asn;
(ii)
(SEQ ID NO: 2)
Arg-Lys-Leu-Met-Gln-Pro-Thr-Arg-Asn-Arg-Arg-
Asn-Pro-Asn-Thr;

(iii) a peptide according to (i) or (ii) wherein at least one of the amino acids is a D-amino acid;
(iv) a functional variant of a peptide according to any one of (i) to (iii); and
(v) a peptide consisting of at least 3-5 contiguous amino acid residues of a peptide according to (i), (ii), (iii) or (iv).

As used herein, the term “Aβ” is intended to encompass naturally occurring proteolytic cleavage products of the Aβ precursor protein (APP) which are involved in Aβ aggregation and/or Aβ-amyloidosis. These peptides include Aβ peptides having 39-43 amino acids such as Aβ₁₋₃₉, Aβ₁₋₄₀, Aβ₁₋₄₁, Aβ₁₋₄₂ and Aβ₁₋₄₃.

As used herein, an “Aβ modulating peptide” is a peptide that, when contacted with Aβ, modulates one or more of Aβ aggregation, Aβ neurotoxicity and peripheral clearance of Aβ.

As used herein the term “Aβ aggregation” refers to a process whereby Aβ peptides associate with each other to form multimeric, largely insoluble complexes and the term “aggregation” encompasses Aβ fibril formation and Aβ plaques.

As used herein, with respect to Aβ modulating peptides associated with Aβ aggregation, the term modulates, modulating and variants thereof encompasses both inhibition and promotion of Aβ aggregation. Aggregation of Aβ is “inhibited” in the presence of the modulator when there is a decrease in the amount and/or rate of Aβ aggregation as compared to the amount and/or rate of Aβ aggregation in the absence of the modulator. The various forms of the term “inhibition” are intended to include both complete and partial inhibition of Aβ aggregation. Inhibition of aggregation can be quantitated using, for example, one or more of (i) the fold increase in the lag time for aggregation; (ii) the decrease in the overall plateau level of aggregation (i.e., total amount of aggregation); or (iii) an assay such as a thioflavin T (ThT) fluorescence assay. Aβ aggregation can also be measured using a gel analysis to visualise “smearing” or atomic force microscopy (AFM) to visualise aggregated Aβ species.

The Aβ modulating peptides which inhibit Aβ aggregation can be used to prevent or delay the onset of Aβ deposition. Preferably, an Aβ modulating peptide of the invention inhibits Aβ aggregation by at least 10%, 20%, 30%, 40%, 50%, 75% or 80% or 90%.

As used herein, with respect to Aβ modulating peptides associated with Aβ neurotoxicity, the term modulates, modulating and variants thereof encompasses partial and complete inhibition of Aβ neurotoxicity. Preferably, the peptides inhibit the formation and/or activity of neurotoxic aggregates of Aβ peptide. Additionally, the peptides preferably reduce the neurotoxicity of preformed Aβ aggregates. In this regard, the peptides of the invention may either bind to preformed Aβ fibrils or soluble aggregate and modulate their inherent neurotoxicity or perturb the equilibrium between monomeric and aggregated forms of Aβ in favour of the non-neurotoxic form.

Inhibition of neurotoxicity can be quantitated using an assay such as a LDH assay that measures the amount of LDH released by cells, a cell viability assay or apoptosis assays (e.g. measurement of caspase activity, which is elevated in apoptotic cells). Preferably, the inhibition of Aβ neurotoxicity is by at least 10%, 20%, 30%, 40%, 50%, 75% or 80% or 90%.

As used herein, with respect to Aβ modulating peptides associated with peripheral clearance of Aβ, the term modulates, modulating and variants thereof encompasses partial and complete peripheral clearance of Aβ from a subject. In this regard, it is believed that agents circulating in the plasma, that are adapted to bind Aβ, are able to extract Aβ via equilibrium in efflux of Aβ across the blood-brain barrier (BBB). The Aβ can then be cleared, for example, via the liver. Peripheral clearance of Aβ can be quantitated using one or more of: measuring reduction in cerebral amyloid deposits and/or one of the methods described in the examples herein.

When the Aβ modulating peptide has at least one D-amino add, it may comprise 1-5, 6-9 or 10-15 D-amino acids. Preferably, all the amino acids in the peptide are D-amino acids.

Functional variants of the present invention include peptides with modified or different amino acids sequences that still retain one or more important characteristics such as their ability to modulate Aβ and/or their ability to bind to Aβ. These functional variants include peptides (such as SEQ ID NO's: 1 and 2) with deletions, insertions, inversions, repeats and/or type substitutions. Preferably, functional variants are at least 70%, 80% or 90% identical to the reference sequence, more preferably at least 95% identical to the reference sequence.

Functional variants also include peptides (i) in which one or more of the amino acid residues are substituted with a conserved or non-conserved amino acid residue such as synthetic, non-naturally occurring analogues and/or natural amino acid residues; or (ii) in which one or more of the amino acid residues includes a substituent group.

Conservative amino acid substitutions are where an amino acid residue is replaced with an amino acid residue having a similar side chain. Particular conserved substitutions involve the substitution of a charged amino acid with an alternative charged amino acid or a negatively charged or neutral amino acid. Other conservative substitutions for the purposes of the present invention are exemplified in Table 1 hereunder where amino acids in a listed group can be substituted. However, it will be appreciated that skilled persons may also determine further conservative substitutions not specifically listed.

TABLE 1

| Group | Amino Acids |
|---|---|
| Aromatic | Phenylalanine, Tryptophan, Tyrosine, Histidine |
| Hydrophobic | Leucine, Isoleucine, Valine, norleucine |
| Small | Alanine, Serine, Threonine, Methionine, Glycine |
| Acidic | Aspartic acid, Glutamic acid |
| Basic | Arginine, Lysine, Histidine |
| Polar | Glutamine, Asparagine |
| Uncharged | Glycine, asparagine, glutamine, serine, |
| Polar | threonine, tyrosine, cysteine |
| Non-polar | Alanine, Valine, Leucine, Isoleucine, |
| | Proline, Phenylalanine, Methionine, Tryptophan |
| Beta- | Threonine, Valine, Isoleucine |
| branched | |

Functional variants may have an enhanced ability to modulate Aβ and/or altered pharmacokinetic properties such as improved stability and include peptides that have been terminally modified. Amino-terminal modifications include the addition of a modifying group comprising a cyclic, heterocyclic, polycyclic or branched alkyl group. Carboxy-terminal modifications include the addition of a peptide amide, a peptide alkyl or aryl amide (e.g., a peptide phenethylamide) or a peptide alcohol. Functional variants also include other modifications such as N-alkyl (or aryl) substitution, or backbone crosslinking to construct lactams and other cyclic structures, C-terminal hydroxymethyl derivatives, O-modified derivatives (e.g., C-terminal hydroxymethyl benzyl ether), N-terminally modified derivatives including substituted amides such as alkylamides and hydrazides.

Cyclic groups include cyclic saturated or unsaturated (i.e., aromatic) group having from about 3 to 10, preferably about 4 to 8, and more preferably about 5 to 7, carbon atoms. Exemplary cyclic groups include cyclopropyl, cyclobutyl, cyclopentyl, cyclohexyl, and cyclooctyl. Cyclic groups may be unsubstituted or substituted at one or more ring positions. Thus, a cyclic group may be substituted with e.g., halogens, alkyls, cycloalkyls, alkenyls, alkynyls, aryls, heterocycles, hydroxyls, aminos, nitros, thiols amines, imines, amides, phosphonates, phosphines, carbonyls, carboxyls, silyls, ethers, thioethers, sulfonyls, sulfonates, selenoethers, ketones, aldehydes, esters, —CF₃, —CN, or the like.

Heterocyclic groups include cyclic saturated or unsaturated (i.e., aromatic) group having from about 3 to 10, preferably about 4 to 8, and more preferably about 5 to 7, carbon atoms, wherein the ring structure includes about one to four heteroatoms. Heterocyclic groups include pyrrolidine, oxolane, thiolane, imidazole, oxazole, piperidine, piperazine, morpholine and pyridine. The heterocyclic ring can be substituted at one or more positions with such substituents as, for example, halogens, alkyls, cycloalkyls, alkenyls, alkynyls, aryls, other heterocycles, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN, or the like. Heterocycles may also be bridged or fused to other cyclic groups as described below.

Polycyclic groups refers to two or more saturated or unsaturated (i.e., aromatic) cyclic rings in which two or more carbons are common to two adjoining rings, e.g., the rings are “fused rings”. Rings that are joined through non-adjacent atoms are termed “bridged” rings. Each of the rings of the polycyclic group can be substituted with such substituents as described above, as for example, halogens, alkyls, cycloalkyls, alkenyls, alkynyls, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN, or the like.

In addition to the cyclic, heterocyclic and polycyclic groups discussed above, other types of modifying groups can be used in a modulator of the invention. For example, hydrophobic groups and branched alkyl groups may be suitable modifying groups. Examples include acetyl groups, phenylacetyl groups, phenylacetyl groups, diphenylacetyl groups, triphenylacetyl groups, isobutanoyl groups, 4-methylvaleryl groups, trans-cinnamoyl groups, butanoyl groups and 1-adamantanecarbonyl groups.

Non-limiting examples of suitable modifying groups and their corresponding modifying reagents are listed in Table 1 below.

TABLE 2

| Modifying Group | Modifying Reagent |
|---|---|
| Cholyl- | Cholic acid |
| Lithocholyl- | Lithocholic acid |
| Hyodeoxycholyl- | Hyodeoxycholic acid |
| Chenodeoxycholyl- | Chenodeoxycholic acid |
| Ursodeoxycholyl- | Ursodeoxycholic acid |
| 3-Hydroxycinnamoyl- | 3-Hydroxycinnamic acid |
| 4-Hydroxycinnamoyl- | 4-Hydroxycinnamic acid |
| 2-Hydroxycinnamoyl- | 2-Hydroxycinnamic acid |
| 3-Hydroxy-4-methoxycinnamoyl- | 3-Hydroxy-4-methoxycinnamic acid |
| 4-Hydroxy-3-methoxycinnamoyl- | 4-Hydroxy-3-methoxycinnamic acid |
| 2-Carboxycinnamoyl- | 2-Carboxycinnamic acid |
| 3-Formylbenzoyl | 3-Carboxybenzaldehyde |
| 4-Formylbenzoyl | 4-Carboxybenzaldehyde |
| 3,4,-Dihydroxyhydrocinnamoyl- | 3,4,-Dihydroxyhydrocinnamic acid |
| 3,7-Dihydroxy-2-napthoyl- | 3,7-Dihydroxy-2-naphthoic acid |
| 4-Formylcinnamoyl- | 4-Formylcinnamic acid |
| 2-Formylphenoxyacetyl- | 2-Formylphenoxyacetic acid |
| 8-Formyl-1-napthoyl | 1,8-napthaldehydic acid |
| 4-(hydroxymethyl)benzoyl- | 4-(hydroxymethyl)benzoic acid |
| 4-Hydroxyphenylacetyl- | 4-Hydroxyphenylacetic acid |
| 3-Hydroxybenzoyl- | 3-Hydroxybenzoic acid |
| 4-Hydroxybenzoyl- | 4-Hydroxybenzoic acid |
| 5-Hydantoinacetyl- | 5-Hydantoinacetic acid |
| L-Hydroorotyl- | L-Hydroorotic acid |
| 4-Methylvaleryl- | 4-Methylvaleric acid |
| 2,4-Dihydroxybenzoyl- | 2,4-Dihydroxybenzoic acid |
| 3,4-Dihydroxycinnamoyl- | 3,4-Dihydroxycinnamic acid |
| 3,5-Dihydroxy-2-naphthoyl- | 3,5-Dihydroxy-2-naphthoic acid |
| 3-Benzoylpropanoyl- | 3-Benzoylpropanoic acid |
| trans-Cinnamoyl- | trans-Cinnamic acid |
| Phenylacetyl- | Phenylacetic acid |
| 2-Hydroxyphenylacetyl- | 2-Hydroxyphenylacetic acid |
| 3-Hydroxyphenylacetyl- | 3-Hydroxyphenylacetic acid |
| Diphenylacetyl- | Diphenylacetic acid |
| Triphenylacetyl- | Triphenylacetic acid |
| (.+−.)-Mandelyl- | (.+−.)-Mandelic acid |
| (.+−.)-2,4-Dihydroxy- | (.+−.)-Pantolactone |
| 3,3-dimethylbutanoyl | |
| Butanoyl- | Butanoic anhydride |
| Isobutanoyl- | Isobutanoic anhydride |
| Hexanoyl- | Hexanoic anhydride |
| Propionyl- | Propionic anhydride |
| 3-Hydroxybutyroyl | beta.-Butyrolactone |
| 4-Hydroxybutyroyl | gamma.-Butyrolactone |
| 3-Hydroxypropionoyl | beta.-Propiolactone |
| 2,4-Dihydroxybutyroyl | alpha.-Hydroxy-.beta.-Butyrolactone |
| 1-Adamantanecarbonyl- | 1-Adamantanecarbonic acid |
| Glycolyl- | Glycolic acid |
| DL-3-(4-hydroxyphenyl)lactyl- | DL-3-(4-hydroxyphenyl)lactic acid |
| 3-(2-Hydroxyphenyl)propionyl- | 3-(2-Hydroxyphenyl)propionic acid |
| D-3-Phenyllactyl- | D-3-Phenyllactic acid |
| Hydrocinnamoyl- | Hydrocinnamic acid |
| 3-(4-Hydroxyphenyl)propionyl- | 3-(4-Hydroxyphenyl)propionic acid |
| L-3-Phenyllactyl- | L-3-Phenyllactic acid |

Preferred modifying groups include biotin-containing groups, fluorescein-containing groups. Functional variants also include derivatives of a peptide in which one or more reaction groups on the peptide have been derivatized with a substituent group. Examples of peptide derivatives include peptides in which an amino acid side chain, the peptide backbone, or the amino- or carboxy-terminus has been derivatized such as peptidic compounds with methylated amide linkages. Chemical modification of one or more residues may be achieved by chemically derivatizing a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

Peptides of the invention may also be stabilised by derivatization using water soluble polymers. The polymer selected should be water soluble so that the peptide to which it is attached does not precipitate in an aqueous environment, such as a physiological environment. The effectiveness of the derivatization may be ascertained by administering the derivative, in the desired form (i.e., by osmotic pump, or, more preferably, by injection or infusion, or, further formulated for oral, pulmonary or nasal delivery, for example), and observing biological effects as described herein.

The water soluble polymer may be selected from the group consisting of, for example, polyethylene glycol, copolymers of ethylene glycol/propylene glycol, carboxymethylcellulose, dextran, polyvinyl alcohol, polyvinyl pyrolidone, poly-1,3-dioxolane, poly-1,3,6-trioxane, ethylene/maleic anhydride copolymer, polyaminoacids (either homopolymers or random copolymers), and dextran or poly(n-vinyl pyrolidone)polyethylene glycol, propylene glycol homopolymers, polypropylene oxide/ethylene oxide co-polymers, polyoxyethylated polyols and polyvinyl alcohol. Polyethylene glycol propionaldehyde may have advantages in manufacturing due to its stability in water. Also, succinate and styrene may also be used.

Other functional variants of the Aβ modulating peptides of the present invention comprise peptides that have been modified to alter the specific properties of the compound while retaining at least one important characteristic such as the ability of the compound to modulate Aβ aggregation, Aβ neurotoxicity or Aβ peripheral clearance. These modifications can be made to alter a pharmacokinetic property, such as in vivo stability, attach a detectable substance/label and/or couple the peptide to an additional therapeutic moiety.

When the Aβ modulating peptides are modified to include a label, suitable labels include various enzymes e.g. horseradish peroxidase, alkaline phosphatase, β-galactosidase, or acetylcholinesterase; prosthetic groups e.g. streptavidinibiotin and avidinibiotin; fluorescent materials e.g. umbelliferone, fluorescein, fluorescein isothiocyanate, rhodamine, dichlorotriazinylamine fluorescein, dansyl chloride or phycoerythrin; luminescent materials e.g. luminal; and radioactive materials e.g. ¹⁴C, ¹²³I, ¹²⁴I, ¹²⁵I, ¹³¹I, ³⁵S or ³H. Other carrier detection molecules such as curcumin or curcumin analogues could also be used to label the Aβ modulating peptides. Labelled peptides can be used to assess the in vivo pharmacokinetics of a peptide and/or to detect Aβ, Aβ aggregation, Aβ neurotoxicity or Aβ peripheral clearance.

When the Aβ modulating peptides are modified to include an additional functional moiety the functional moiety may be varied and includes a compound capable of breaking down or dissolving amyloid plaques or otherwise disrupting Aβ aggregation.

Pharmaceutical Compositions

Peptides of the invention may be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline.

In one embodiment, the compositions include an Aβ modulating peptide in a therapeutically or prophylactically effective amount sufficient to modulate Aβ aggregation, Aβ neurotoxicity or Aβ peripheral clearance and a pharmaceutically acceptable carrier. A “therapeutically effective amount” refers to an amount effective, at dosages and for periods of time necessary, to achieve the desired therapeutic result, such as reduction or reversal or Aβ deposition or Aβ neurotoxicity, an increase in Aβ peripheral clearance and/or treat an amyloid disease.

The therapeutically effective amount of modulator may vary according to factors such as the disease state, age, sex, and weight of the individual, and the ability of the modulator to elicit a desired response in the individual. Dosage regimens may be adjusted to provide the optimum therapeutic response. A therapeutically effective amount is also one in which any toxic or detrimental effects of the modulator are outweighed by the therapeutically beneficial effects.

A “prophylactically effective amount” refers to an amount effective, at dosages and for periods of time necessary, to achieve the desired prophylactic result, such as preventing or inhibiting the rate of Aβ deposition and/or Aβ neurotoxicity in a subject predisposed to Aβ deposition or an amount that that reduces progression between two disease stages selected from the stages of (i) preclinical amyloidosis (ii) mild cognitive impairment (MCI) and (iii) amyloid (e.g. Alzheimer's disease) mediated dementia. A prophylactically effective amount can be determined as described above for the therapeutically effective amount. Typically, since a prophylactic dose is used in subjects prior to or at an earlier stage of disease, the prophylactically effective amount will be less than the therapeutically effective amount.

One factor that may be considered when determining a therapeutically or prophylactically effective amount of an Aβ modulating peptide is the concentration of natural Aβ in a biological compartment of a subject, such as in the cerebrospinal fluid (CSF) of the subject. A non-limiting range for a therapeutically or prophylactically effective amount of an Aβ modulating peptide is 0.01 nM-10 μM. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The amount of active compound in the composition may vary according to factors such as the disease state, age, sex, and weight of the individual, each of which may affect the amount of natural Aβ in the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. See, for example, Remington's Pharmaceutical Sciences, 19th Ed. (1995, Mack Publishing Co., Easton, Pa.) which is herein incorporated by reference.

The preferred form of the pharmaceutical composition depends on the intended mode of administration and therapeutic application. Pharmaceutical compositions prepared according to the invention may be administered by any means that leads to the peptides of the invention coming in contact with a causative agent of a disease or disorder as herein described.

When treating a subject with an amyloid disease, such as Alzheimer's disease, a mode of administration will be through such routes of administration as intra cerebroventricular, parenteral, intramuscular, intravenous, subcutaneous, intraocular delivery, oral or transdermal administration by means of a syringe, optionally a pen-like syringe, or intra nasal, buccal and transdermal patch. Such administration will desirably may be by injection. Parenteral administration may also be used to introduce pharmaceutical compositions into a patient. In an alternative form of the invention the pharmaceutical composition can be administered by means of an infusion pump.

In another embodiment, a pharmaceutical composition comprising an Aβ modulating peptide of the invention is formulated such that the Aβ modulating peptide is transported across the blood-brain barrier (BBB). Various strategies for increasing transport across the BBB can be adapted to the peptides of the invention to thereby enhance their transport across the BBB. In one approach, the Aβ modulating peptide can be chemically modified to form a prodrug with enhanced transmembrane transport. Suitable chemical modifications include covalent linking of a fatty acid to the modulator through an amide or ester linkage and glycating the modulator. Also, N-acylamino acid derivatives may be used in a modulator to form a “lipidic” prodrug.

In another approach for enhancing transport across the BBB, a peptidic or pepticlomimetic modulator is conjugated to a second peptide or protein, thereby forming a chimeric protein, wherein the second peptide or protein undergoes absorptive-mediated or receptor-mediated transcytosis through the BBB. Accordingly, by coupling the modulator to this second peptide or protein, the chimeric protein is transported across the BBB. The second peptide or protein can be a ligand for a brain capillary endothelial cell receptor ligand. For example, a preferred ligand is a monoclonal antibody that specifically binds to the transferrin receptor on brain capillary endothelial cells. Other suitable peptides or proteins that can mediate transport across the BBB include histones and ligands such as biotin, folate, niacin, pantothenic acid, riboflavin, thiamin, pyridoxal and ascorbic acid. Additionally, the glucose transporter GLUT-1 is capable of transporting glycopeptides across the BBB. Chimeric proteins can be formed by recombinant DNA methods (e.g., by formation of a chimeric gene encoding a fusion protein) or by chemical crosslinking of the modulator to the second peptide or protein to form a chimeric protein. Numerous chemical crosslinking agents are known and a crosslinking agent can be chosen which allows for high yield coupling of the Aβ modulating peptide to the second peptide or protein and for subsequent cleavage of the linker to release bioactive agent.

In yet another approach for enhancing transport across the BBB, the Aβ modulating peptide is encapsulated in a carrier vector which mediates transport across the BBB. For example, the modulator can be encapsulated in a liposome, such as a positively charged unilamellar liposome or in polymeric microspheres. Moreover, the carrier vector can be modified to target it for transport across the BBB. For example, the carrier vector (e.g., liposome) can be covalently modified with a molecule which is actively transported across the BBB or with a ligand for brain endothelial cell receptors, such as a monoclonal antibody that specifically binds to transferrin receptors.

In still another approach to enhancing transport of the modulator across the BBB, the Aβ modulating peptide is co-administered with another agent which functions to permeabilize the BBB. Examples of such BBB “permeabilizers” include bradykinin and bradykinin agonists. Other examples include agents (ie small interfering RNA, siRNA) designed to periodically and reversibly modulate the tight junctions of the BBB. This allows for a size-selective tight junction to be established where by passive diffusion of molecules across their own concentration gradient can occur.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the peptide. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy use with a syringe exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars such as mannitol or dextrose or sodium chloride.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Additionally, auxiliary substances, such as wetting or emulsifying agents, surfactants, pH buffering substances and the like can be present in compositions. Other components of pharmaceutical compositions are those of animal, vegetable, or synthetic origin oils, for example, peanut oil, soybean oil, and mineral oil. In general, glycols such as propylene glycol or polyethylene glycol are preferred liquid carriers, particularly for injectable solutions.

Additional formulations suitable for other modes of administration include oral, intranasal, and pulmonary formulations, suppositories, and transdermal applications.

The routes of administration described herein are intended only as a guide since a skilled practitioner will be able to determine readily the optimum route of administration and dosage for any particular patient.

Methods of Modulating Aβ Aggregation, Aβ Neurotoxicity and/or Aβ Peripheral Clearance

Another aspect of the invention provides a method for modulating Aβ aggregation, Aβ neurotoxicity and/or Aβ peripheral clearance comprising the step of contacting Aβ with an Aβ modulating peptide of the present invention.

Methods of Treatment

Since the Aβ modulating peptides of the invention can modulate Aβ aggregation, Aβ neurotoxicity and/or Aβ peripheral clearance, the peptides are also useful in the treatment of disorders associated with amyloidosis, either prophylactically or therapeutically. With respect to prophylactic and/or therapeutic use, it will be appreciated that the outcome may be to slow, stop or otherwise affect, in a positive fashion, the progression of amyloid disease, such as Alzheimer's disease. Where progression includes but is not limited to progression between any two disease stages such as (i) preclinical amyloidosis (ii) mild cognitive impairment (MCI) and (iii) amyloid (e.g. Alzheimer's disease) mediated dementia Accordingly, another use of the peptides of the invention is as therapeutic agents to modulate Aβ aggregation, Aβ neurotoxicity and/or Aβ peripheral clearance.

Thus, in another embodiment, the invention provides a method for modulating Aβ aggregation, Aβ neurotoxicity and/or Aβ peripheral clearance, which can be used prophylactically or therapeutically in the treatment or prevention of disorders associated with amyloidosis. Aβ modulating peptides of the invention can reduce the toxicity of Aβ aggregates to neuronal cells. Moreover, the peptides may have the ability to reduce the neurotoxicity of preformed Aβ fibrils or oligomers. Accordingly, the Aβ modulating peptides of the invention can be used to inhibit or prevent the formation of neurotoxic Aβ oligomers and/or fibrils in subjects (e.g., prophylactically in a subject predisposed to Aβ deposition) and can be used to reverse amyloidosis therapeutically in subjects already exhibiting Aβ deposition.

For the purpose of the present invention “amyloid diseases” or “amyloidoses” include a number of disease states having a wide variety of outward symptoms. These disorders have in common the presence of abnormal extracellular deposits of protein fibrils, known as “amyloid deposits” or “amyloid plaques”. Amyloid diseases include, without limitation, such disease states as (a) AA amyloidosis (caused by such ailments as chronic inflammatory disorders (e.g. rheumatoid arthritis, juvenile chronic arthritis, ankylosing spondylitis, psoriasis, psoriatic arthropathy, Reiter's syndrome, Adult Still's disease, Behcet's syndrome, and Crohn's disease), chronic local or systemic microbial infections (e.g. such as leprosy, tuberculosis, bronchiectasis, decubitus ulcers, chronic pyelonephritis, osteomyelitis, and Whipple's disease), and malignant neoplasms (e.g. Hodgkin's lymphoma, renal carcinoma, carcinomas of gut, lung and urogenital tract, basal cell carcinoma, and hairy cell leukaemia); (b) AL Amyloidoses which is generally associated with almost any dyscrasia of the B lymphocyte lineage, ranging from malignancy of plasma cells (multiple myeloma) to benign monoclonal gammopathy; (c) hereditary systemic amyloidoses; (d) Senile Systemic Amyloidosis; (e) Cerebral Amyloidosis; (f) Dialysis-related Amyloidosis (g) Hormone-derived Amyloidoses and (h) Miscellaneous Amyloidoses that are normally manifest as localized deposits of amyloid (such as idiopathic deposition include nodular AL amyloid, cutaneous amyloid, endocrine amyloid, and tumour-related amyloid).

The invention also extends to the treatment of Inclusion-Body Myositis (IBM). IBM is a progressive and debilitating muscle disease usually of persons over 50 years of age. It is of unknown cause and there is no successful treatment. Interestingly, however, there are remarkable similarities between IBM muscle pathology and the AD brain. These include the abnormal accumulation, misfolding, and aggregation of Aβ; accumulation of APP, phosphorylated tau and other Alzheimer- and dementia-related proteins including presenilin, prion protein and α-synuclein; and the accumulation of cholesterol, apolipoprotein E and low-density lipoprotein receptors. It is now known that increased SOD-like activity and free radical toxicity are important in IBM pathogenesis. Given the role of oxidative stress in the progression of both AD and IBM, new treatments that target the oxidative injury process in AD are also likely to be effective in treating IBM.

Aβ modulating peptides of the invention can be contacted with Aβ present in a subject (e.g., in the cerebrospinal fluid or cerebrum of the subject) to thereby modulate Aβ aggregation, Aβ neurotoxicity and/or Aβ peripheral clearance. An Aβ modulating peptide alone can be administered to the subject, or alternatively, the peptide can be administered in combination with other therapeutically active agents (e.g., as discussed above). When combination therapy is employed, the therapeutic agents can be coadministered in a single pharmaceutical composition, coadministered in separate pharmaceutical compositions or administered sequentially.

The Aβ modulating peptide may be administered to a subject by any suitable route effective for inhibiting Aβ aggregation in the subject, although in a particularly preferred embodiment, the Aβ modulating peptide is administered parenterally, most preferably to the central nervous system of the subject. Possible routes of CNS administration include intraspinal administration and intracerebral administration (e.g., intracerebrovascular administration). Alternatively, the peptides can be administered, for example, orally, intraperitoneally, intravenously or intramuscularly. For non-CNS administration routes, the Aβ modulating peptide can be administered in a formulation which allows for transport across the BBB. Certain peptides may be transported across the BBB without any additional further modification whereas others may need further modification as described above.

Suitable modes and devices for delivery of therapeutic compounds to the CNS include cerebrovascular reservoirs, catheters for intrathecal delivery, injectable intrathecal reservoirs, implantable infusion pump systems and osmotic pumps.

The method of the invention for modulating Aβ aggregation, Aβ neurotoxicity and/or Aβ peripheral clearance in vivo, can be used therapeutically in diseases associated with abnormal Aβ aggregation and deposition to thereby slow the rate of Aβ deposition and/or lessen the degree of Aβ deposition, thereby ameliorating the course of the disease. In a preferred embodiment, the method is used to treat Alzheimer's disease (e.g., sporadic or familial AD, including both individuals exhibiting symptoms of AD and individuals susceptible to familial AD). The method can also be used prophylactically or therapeutically to treat other clinical occurrences of Aβ deposition, such as in Down's syndrome individuals and in patients with hereditary cerebral haemorrhage with amyloidosis-Dutch-type (HCHWA-D). While inhibition of Aβ aggregation, Aβ neurotoxicity and/or enhancement of Aβ peripheral clearance is a preferred therapeutic method, Aβ modulating peptides that promote Aβ aggregation may also be useful therapeutically by allowing for the sequestration of Aβ at sites that do not lead to neurological impairment.

Additionally, abnormal accumulation of Aβ precursor protein (APP) in muscle fibres has been implicated in the pathology of sporadic inclusion body myositis (IBM). Accordingly, the Aβ modulating peptides of the invention can be used prophylactically or therapeutically in the treatment of disorders in which Aβ, or APP, is abnormally deposited at non-neurological locations, such as treatment of IBM by delivery of the peptides to muscle fibres.

Detection Methods

Since the Aβ modulating peptides of the present invention interact with Aβ, they can be used to detect Aβ, either in vitro or in vivo. Thus, another embodiment of the present invention is the use of the Aβ modulating peptides of the invention as agents to detect the presence of Aβ, either in a biological sample or in viva in a subject. Furthermore, detection of Aβ, utilizing a modulating peptide of the invention, can be used to diagnose amyloidosis in a subject.

Thus, another embodiment of the invention provides a method for detecting Aβ comprising the step of contacting a sample with an Aβ modulating peptide of the present invention and detecting the formation of a complex between the Aβ and the Aβ modulating peptide.

The method of detection can be to detect and quantitate Aβ in sample (e,g., a sample of biological fluid). To aid in detection, the Aβ modulating peptide can comprise a detectable substance.

The sample can be from any biological fluid capable of carrying Aβ and includes cerebrospinal fluid. Preferably, the sample is contacted with Aβ modulating peptide of the invention and the amount of Aβ is then measured by a suitable assay, such as by the assays described in the examples herein. The amount of Aβ and/or its degree of aggregation in the sample can be compared to that of a control sample(s) of a known concentration of Aβ, similarly contacted with the modulator and the results can be used as an indication of whether a subject is susceptible to or has a disorder associated with amyloidosis. The Aβ can also be detected by detecting the detectable substance incorporated into the Aβ modulating peptide. Examples of detectable substances include biotin (e.g., an amino-terminally biotinylated Aβ modulating peptide) can be detected using a streptavidin or avidin probe which is labelled with a detectable substance (e.g., an enzyme, such as peroxidase).

In viva methods include the use of Aβ modulating peptide to detect, and, if desired, quantitate, Aβ in a subject, for example to aid in the diagnosis of amyloidosis in the subject. To aid in detection, the modulator compound can be modified with a detectable substance, such as ⁹⁹mTc or radioactive iodine, which can be detected it, vivo in a subject. The labelled Aβ modulating peptide can be administered to the subject and, after sufficient time to allow accumulation of the peptide at sites of amyloid deposition, the labelled modulator compound can be detected by suitable imaging techniques. When a radioactive label is used, the radioactive signal can be directly detected (e.g., whole body counting), or alternatively, the radioactive signal can be converted into an image on an autoradiograph or on a computer screen to allow for imaging of amyloid deposits in the subject. Suitable radioactive labels include iodine such as ¹²³I, ¹²⁴I, ¹²⁵I and ¹³¹I. Test include one or more of brain or whole body scintigraphy, positron emission tomography (PET), metabolic turnover studies and brain or whole body counting and delayed low resolution imaging studies.

Thus, the present invention also provides a method for detecting Aβ to facilitate diagnosis of a Alzheimer's disease, comprising contacting a biological sample with an Aβ modulating peptide of the invention and detecting the peptide bound to Aβ to facilitate diagnosis of an amyloidogenic disease. In one embodiment, the modulating peptide and the biological sample are contacted in vitro. In another embodiment, the Aβ modulating peptide is contacted with the biological sample by administering the peptide to a subject.

Nucleotides

The present invention also provides polynucleotides encoding the peptides of the invention. It will be understood by a skilled person that due to the degeneracy of the amino acid code, numerous different polynucleotides can encode the same peptide as a result of the degeneracy of the genetic code. In addition, it is to be understood that skilled persons may, using routine techniques, make nucleotide substitutions that do not affect the peptide sequence encoded by the polynucleotides of the invention to reflect the codon usage of any particular host organism in which the polypeptides of the invention are to be expressed.

Polynucleotides of the invention may comprise DNA or RNA. They may be single-stranded or double-stranded. They may also be polynucleotides that include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynuclectides of the invention.

Where the polynucleotide of the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however be understood to be common general knowledge.

Manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

This invention is further illustrated by the following examples which should not be construed as limiting. A modulator's properties described below are predictive of the modulators ability to perform the same function in viva. The contents of all references, patents and published patent applications cited throughout this application are hereby incorporated by reference.

### EXAMPLE 1

### Peptide Stability

Materials/Methods

Peptides were prepared as a 1 mM solution in phosphate-buffered saline. 20 μl of the peptide solution was diluted in 10% rat brain homogenate (in 1× phosphate-buffered saline and 0.5% Triton X-100).

The solution was incubated at 37° C. for different times, and the reaction was stopped by adding the Complete mixture of protease inhibitors (Roche Molecular Biochemicals, Mannheim, Germany). For ANA5 and RI-ANA5, the bulk of the brain proteins (but not the peptides) were precipitated in cold methanol (1:4 (v/v) mixture/MeOH) for 1 hr at −20° C. The precipitated proteins were pelleted by centrifugation at 10,000 g for 10 min at 4° C. The supernatant containing the peptide was concentrated five times under vacuum and separated by RP-HPLC. Due to recovery issues with the longer ANA1 and RI-ANA1 peptides, samples were instead lyophilized and then reconstituted in a TFA solution, prior to separation by RP-HPLC.

The area of the peak (UV absorbance at 205 nm) corresponding to the intact peptide was measured and compared with an equivalent sample incubated in phosphate-buffered saline.

In Table 3 below (and elsewhere herein) please note:
ANA1=Thr-Asn-Pro-Asn-Arg-Arg-Asn-Arg-Thr-Pro-Gln-Met-Leu-Lys-Arg
ANA5=Asn-Arg-Thr-Pro-Gln-Met-Leu-Lys-Arg
RI-ANA1=Arg-Lys-Leu-Met-Gln-Pro-Thr-Arg-Asn-Arg-Arg-Asn-Pro-Asn-Thr (SEQ ID NO:2) where all amino acids are D-amino acids
RI-ANA5=Arg-Lys-Leu-Met-Gln-Pro-Thr-Arg-Asn (SEQ ID NO:1) where all amino acids are D-amino acids

Results

TABLE 3

| Time | ANA1 | RI-ANA1 | ANA5 | RI-ANA5 |
|---|---|---|---|---|
| (min) | (μg) | (μg) | (μg) | (μg) |
| 0 | 25.8 | 29.8 | 12 | 14 |
| 15 | 5.6 | 27.8 | — | — |
| 30 | 0.03 | 29.4 | 0 | — |
| 300 | 0.02 | 33.5 | — | 12.5 |

### EXAMPLE 2

### Aggregation of Aβ

Materials/Methods

ThT Time Course Assay

Aβ peptide is solubilised in HFIP to remove any secondary structure; upon evaporation of the HFIP, dry films are stored at −80° C. until use. Aβ is prepared as a monomeric (unaggregated) stock by dissolving to 5 mM concentration in dry DMSO, vortexing for 30 s and incubating in a sonic water bath for 5-10 min. For the time course assay, Aβ (2 uM) was incubated in Tris-buffered saline (TBS) in the presence of 10 uM ThT and the presence/absence of the test peptides in a black-walled, clear base 96 well microplate at a total volume of 100 uL. Samples in the plate were incubated at 37° C., with shaking for 30 s prior to each reading (10 min intervals) using a FLUOstar Optima Plate Reader (excitation wavelength=450 nm: emission wavelength=482 nm; gain=1200). Samples were assayed in triplicate and the blank ThT fluorescence was subtracted from each reading.

ThT Time Course Assay (spiked)

For assays involving spiking of samples, the same protocol as described above was used but modified to include spiking of samples 2 h after commencement of the assay.

ThT Endpoint Assay

Aβ (5 mM in DMSO, as per (i) above) was incubated under conditions that promote its oligomerisation (dilution to 100 uM in cold F12 medium, 24 h, 4° C.) in the presence/absence of the test peptides. These samples were clarified by centrifugation (14000 rpm, 1 min, 4° C.) and the supernatant (9.8 uL=4.5 ug) was incubated with ThT (200 uM in 50 mM Glycine-NaOH; pH 8.5) for 3-5 min. The fluorescence was read using a FLUOstar Optima Plate Reader (25° C.; excitation wavelength=450 nm: emission wavelength=482 nm; gain-adjusted to highest reading). Samples were assayed in triplicate and the blank ThT fluorescence was subtracted from each reading, as for the ThT Time course Assay above.

Results

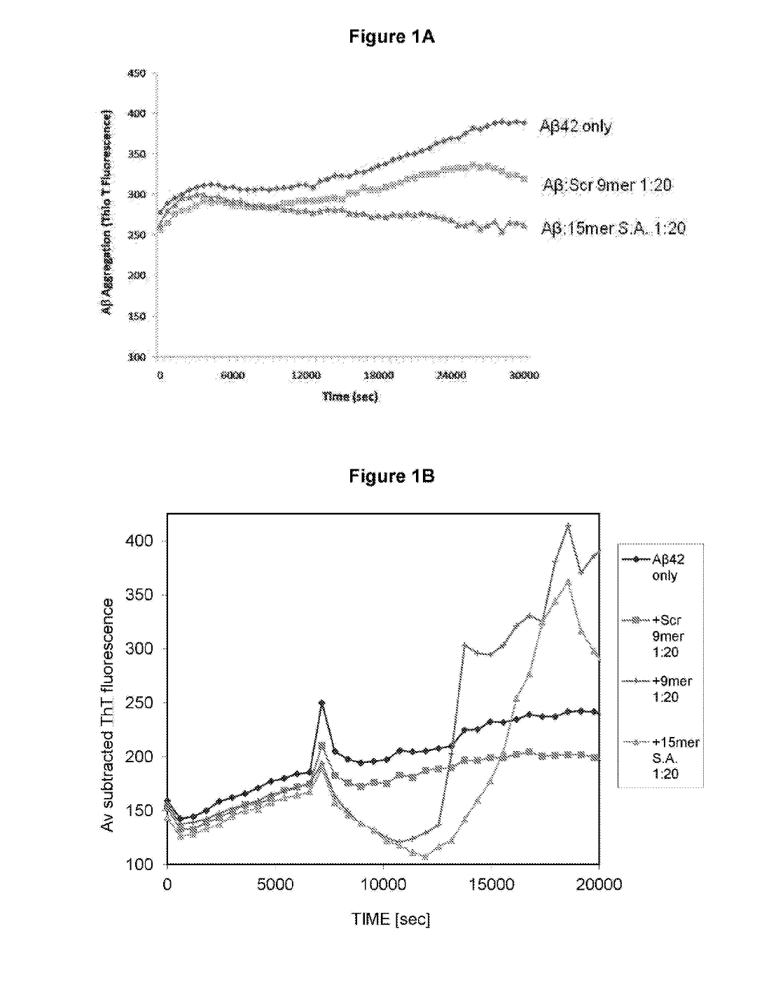

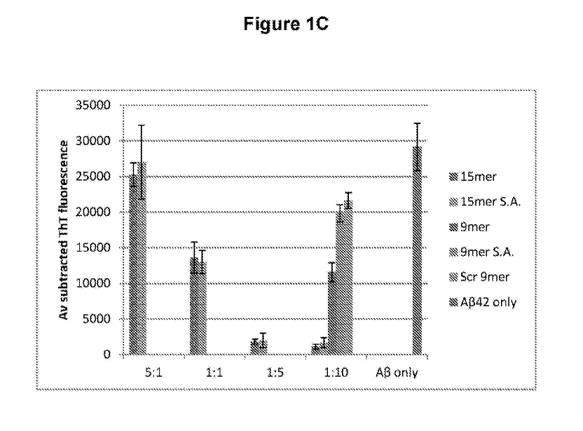

The results are illustrated in FIGS. 1A, 1B and 1C.

### EXAMPLE 3

### Oligomerisation of Aβ

Materials/Methods

Neurotoxicity Assay

M17 neuroblastoma cells were plated in 48-well plates with 25,000 cells in 500 ul media (DMEM/F12 1:1 with 10%FCS) per well, overnight. Cells, at 50-60% confluence, were then treated with Aβ42 in the presence or absence of the peptides and incubated at 37° C., 5% CO₂ for 4 days. On the fourth day, the evaluation of neurotoxicity was performed using the LDH assay (CytoTox-ONE™ Homogeneous Membrane Integrity Assay, Promega) and the MTS assay (CellTiter96® Aqueous One Solution Cell Proliferation Assay, Promega), according to the manufacture's recommendations. Samples were assayed in triplicate.

Gel Analysis (SDS-PAGE and Western Immunoblotting)

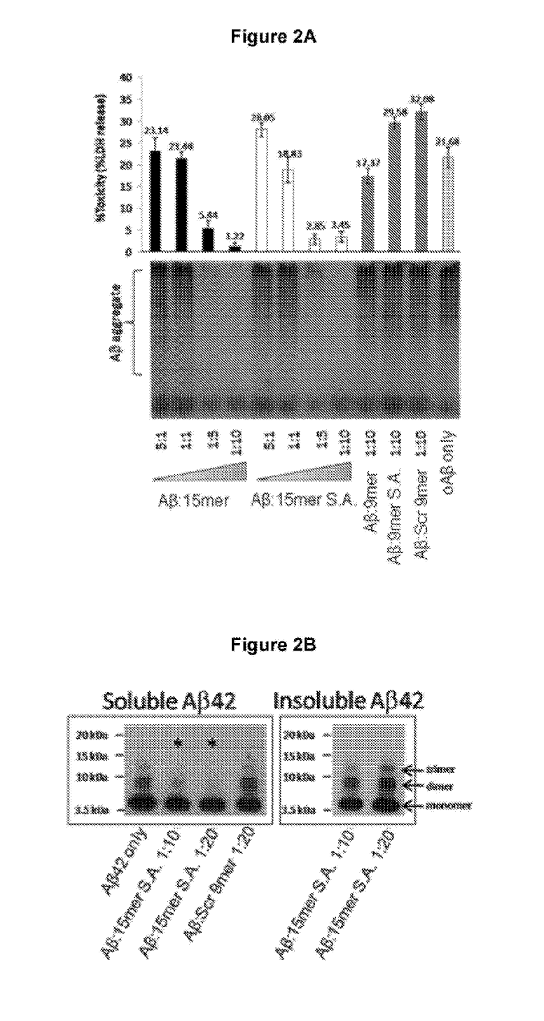

Samples for gel analysis were prepared in 2 ways, i.e. denaturing and non-denaturing condition. Non-denatured samples were used to partially preserve the Aβ aggregates in the samples. For this purpose, samples underwent electrophoresis using the Blue Native Page (BN-PAGE) system. Samples were diluted in MOPS loading buffer (50 mM MOPS, 50 mM Tris, 20% Glycerol, 0.05% Coomasie, pH 77) without reducing agent and were not-heat denatured (FIG. 2A). As the insoluble material was seen in the Aβ preparation, samples also were analysed under denaturing conditions to solubilise this material. In the denaturing condition, samples were diluted in SDS-PAGE loading buffer plus reducing agent (NuPAGE®), and were heat-denatured (72° C., 10 min) prior to loading on polyacrylamide gels (FIG. 2B). Following separation of proteins by SDS-PAGE using NuPAGE® Novex 4-12% Bis-Tris gradient gels, samples were transferred onto, either nitrocellulose (for denatured and semi-denatured samples) or PVDF (non-denatured samples) membranes using iBlot® blotting system (Life Technology). Samples were subjected to Western Immunoblotting with WO2 antibody, to detect Aβ species. Bands were visualised using enhanced chemiluminescence detection and exposure to X-Ray film.

Atomic Force Microscopy (AFM)

Aβ samples were spotted on the freshly cleaved grade V1 muscovite mica and incubated for 5 minutes. Samples were then rinsed with 0.22 μm syringe-filtered double-distilled water and blow dried with several gentle pulses of compressed air (N₂). Samples were then visualized under the AFM (NT-MDT) using semi-contact mode with these following parameters: a minimum contact force, amplitude between 0.5-2V (depending on the cantilever) to generate magnitude around 20 nA, and scan rates about 0.5-1 Hz. All data were processed using Nova NT-MDT Software v1.1.0.1780.

Results

The results are illustrated in FIGS. 2A and 2B. ANA1 (15 mer) and RI-ANA1 (15 mer S.A.) caused a dose-dependent decrease in ThT fluorescence and Aβ aggregation, as measured by the ThT endpoint assay. ANA5 resulted in a similar effect, but with reduced potency (1:1 ratio of Aβ: ANA1/RI-ANA1 was similar in effect to 1:10 ratio of Aβ: ANA5). Although it was stable, the RI-ANA5 peptide was markedly reduced in potency and overlapped with the results obtained using the scrambled ANA5 control peptide as the same molar ratio. The neurotoxicity results echoed the findings from the ThT assay the ANA1 and RI-ANA1 produced a dose-dependent decrease in Aβ neurotoxicity, ANA5 performed less potently, but still with measurable effect; RI-ANA5 did not render a measurable effect at the ratio tested. Reduction in neurotoxicity correlated with a decrease in the Aβ aggregation “smear” in the soluble fraction detected by SOS-PAGE and Western Immunoblotting (FIG. 2A), but an increase in Aβ species in the insoluble fraction (FIG. 2B). Thus, it appeared that RI-ANA1 resulted in the formation of non-toxic, large Aβ aggregates when present at effective molar ratios during conditions favouring Aβ oligomerisation.

The results of further analysis of the protein mixtures from FIG. 2B using atomic force microscopy (AFM) are set out in Table 4 below. These indicate that the presence of RI-ANA1 during Aβ oligomerisation results in the formation of large aggregates (25-30 nm c.f. Aβ only 1.5-4 nm diameter). These are non-toxic, as they were resuspended back into solution prior to dilution and addition to M17 cells in the neurotoxicity assay and did not result in loss of cell viability, as measured by the LDH assay.

TABLE 4

| Sample | Result (physical characteristics of pellet/supernatant) |
|---|---|
| Aβ42 only | Main population: diameter 1.5-4 nm |
| | Other: few larger aggregates 8-12 nm |
| Aβ42 + RI-ANA1 | Main population: diameter 1.5-4 nm |
| 1:20 (supernatant) | Other: few larger aggregates 8-12 nm |
| Aβ42 + RI-ANA1 | Small aggregates: 2-6 nm |
| 1:20 (pellet and | Medium aggregates: 15 nm (average) |
| supernatant) | Extreme aggregates: 25-30 nm |

### EXAMPLE 4

### Effects on Pre-Oligomerised Aβ

Materials/Methods

Gel Analysis and Western Immunoblotting as detailed in Example 3 were used to assess the effects of combining peptides of the invention with pre-oligomerised Aβ and incubating for 4 days at 37° C., with sampling each day from day 0 (d0) to day 4(d4). However, here samples were diluted in SDS-PAGE loading buffer, with no reducing agent, and were not heat-denatured prior to loading on gels, to maintain some structure in the Aβ assemblies, but still allow proteins to be separated on the basis of size.

Results

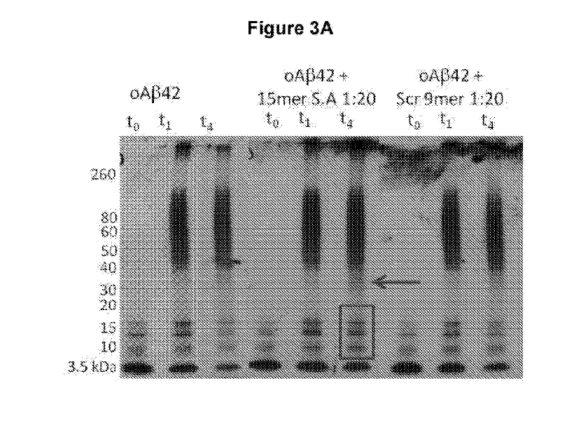

The results are illustrated in FIG. 3A.

### EXAMPLE 5

### Clearance of Aβ42 from Plasma

Materials/Methods

Animals

Our colony of APOE knock-in mice homozygous (targeted replacement) for human APOEε4, as described previously [Sullivan et al., 1997], were derived from animals sourced from Taconic (Germantown, N.Y., USA). APOE knock-out mice (B6.129P2 ApoE-/-, were originally obtained from the Jackson Laboratory, Bar Harbor, Me.). All mice were bred and maintained at the Animal Resources Centre (ARC, Perth, Western Australia). Mice were housed 5-6 per cage in a controlled environment at 22° C. on a 12 h day/night cycle (light from 0700 to 1900 h). A standard laboratory chow diet (Rat and Mouse Cubes, Specialty Feeds Glen Forrest, WA, Australia) and water were consumed ad libitum. This study was conducted in accordance with the Australian code of practice for the care and use of animals for scientific purposes as specified by the National Health and Medical Research Council (NHMRC). The experimental protocols were approved by the University of Western Australia Animal Ethics Committee.

Preparation of Aβ42 and RI-ANA1 peptide solutions Human synthetic Aβ42 peptide was purchased from the W. M. Keck Foundation Biotechnology Resource Laboratory (Yale University, New Haven, Conn.) Stock Aβ42 was prepared by dissolving the Aβ42 peptide in 10% Dimethyl sulfoxide (DMSO) to a concentration of 1 mg/ml. The stock was diluted in sterile isotonic saline solution immediately before experimentation to a concentration of 20 μg in 50 μL. This preparation method yields a consistently predominantly monomeric Aβ42 preparation (Sharman et al., 2010). RI-ANA1 peptide used in this study was obtained from Mimotopes, Australia.

Antibodies

Monoclonal WO2 antibody raised against amino acid residues 5 to 8 of the Aβ domain was generously provided by Professor Konrad Beyreuther (University of Heidelberg, Heidelberg, Germany).

Sampling of Plasma and Liver Aβ42 Levels

To examine any effects of RI-ANA1 in the peripheral clearance of Aβ42, 12 month old human APOEε4 knock-in mice (targeted replacement) were anaesthetized with an intraperitoneal injection of Ketamine/Xylazine (75/10 mg/kg). Mice were divided in three different groups and injected with Aβ42 peptide (20 μg/50 μL), Aβ42 peptide (20 μg/50 μL) plus 0.5 mg and 1 mg of RI-ANA1 respectively via the lateral tail vein. Blood was collected over a 30 min period. Blood samples were taken from the retro-orbital sinus using 1.0 mm diameter heparinised haematocrit tubes at 2.5, 5, 10, 20 and 30 min post-injection for Aβ analysis. Plasma samples were collected after the whole blood was centrifuged at 2000 g for 15 min at 4° C. Mice were sacrificed at 30 minutes post-injection via cardiac puncture. Liver tissue was collected and processed for subsequent analysis of Aβ42 levels.

Analysis of Plasma and Liver Aβ42 Content

Plasma (1 μl) and liver tissue samples (75 μg total protein) were loaded onto 4-12% Bis/Tris NuPAGE® Novex® Mini Gels (Invitragen, USA) with MES buffer and separated for 2.5 h at 90V. The proteins were then transferred to nitrocellulose membranes using the iBlot™ Dry Blotting System (Invitrogen, USA) for 8 min at 20V and immunoblotted. WO2 antibody (1:2,000 dilution), was incubated with membranes for 2 h at room temperature in Tris-buffered saline Tween-20 (TBST), pH 7.4 with 0.5% (w/v) skim milk. HRP-linked goat anti-mouse IgG (1:5,000 dilution) was incubated with membranes for 1 h at room temperature in TBST, pH7.4 with 0.5%(w/v) skim milk. Protein visualization was achieved using enhanced chemiluminescence (ECL) western blotting detection reagents and exposure to hyperfilm-ECL film (GE Healthcare Bio-Sciences, Rydalmere, NSW, Australia). The ECL films were then scanned for densito-metric analysis.

Statistical Analyses

Means and standard deviations were calculated for all variables using conventional methods. A repeated measures design and one-way ANOVA was used to evaluate significant differences amongst the different groups. A criterion alpha level of P<0.05 was used for all statistical comparisons. All data were analysed using SPSS version 19.0 (SPSS, Chicago, Ill., USA).

Results

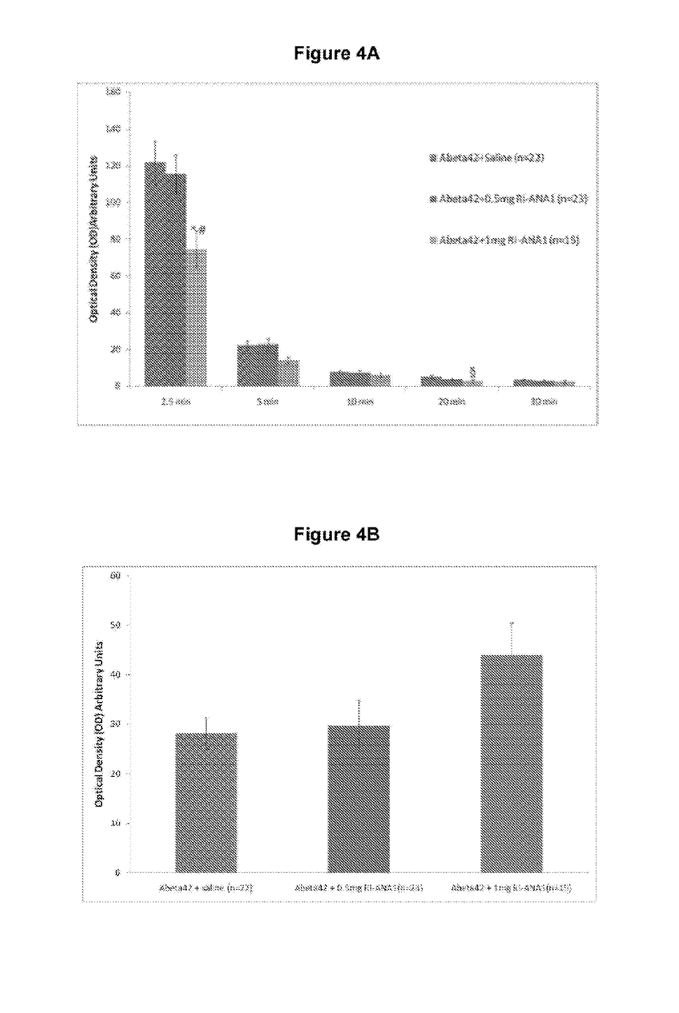

The results are illustrated in FIGS. 4A and 4B.

The levels of injected Aβ42 in the plasma rapidly decrease from 2.5 min post injection, to nearly undetectable levels at 60 min post injection. Compared to Aβ42 injected mice, at 2.5 min post-injection, a significant reduction is observed in those mice injected with Aβ42 +1 mg RI-ANA1 (FIG. 4A). Although not statistically significant, analysis of Aβ42 present in the liver at 60 min (FIG. 4B) demonstrates a trend towards an increased uptake/retention in presence of 1 mg RI-ANA1 (p=0.07).

### EXAMPLES 6A-6G

General Materials/Methods

(i) Peptides: Human synthetic Aβ42 peptide was purchased from the W. M. Keck Foundation Biotechnology Resource Laboratory (Yale University, New Haven, Conn.). All other unlabelled peptides used in this study were obtained from Mimotopes, Melbourne, Australia:
ANA-1=TNPNRRNRTPQMLKR
RI-ANA1=RKLMQPTRNRRNPNT where all amino acids are D-amino acids (also referred to herein as 15M S.A.)
ANA5=NRTPQMLKR
RI-ANA5=RKLMQPTRN where all amino acids are D-amino acids (also referred to herein as 9M S.A.
CTL1=scrambled control based on ANA5,
CTL1 S.A=CTL1 where all amino acids are D-amino acids,
CTL2 S.A.=all amino acids are D-amino acids (stable analogue control based on unrelated APP 9 mer fragment

Tetramethyl rhodamine (TMR)-labelled RI-ANA1, and CTL2 S.A. were also obtained from Mimotopes (Melbourne, Australia). Tritium-labelling of RI-ANA1 peptide was performed by American Radiolabeled Chemicals, Inc. (St. Louis, Mo.).

(ii) Preparation of Aβ42 monomers, oligomers and fibrils—Aβ42 assemblies were prepared according to the methods described in Stine, W. B. at al (2011). Briefly, Aβ42 was solubilised in 1,1,1,3,3,3-hexafluoro-2-propanol (SIGMA), dried and reconstituted in dry dimethyl sulfoxide (SIGMA) to 5 mM concentration. For monomeric Aβ42, the 5 mM stock was diluted to 100 μM in Milli-Q water and used immediately. For oligomeric and fibrillar Aβ42, the 5 mM stock was diluted to 100 μM in either ice-cold Ham's F12 media (C-72110, PromoCell GmbH, Germany) or 10 mM HCl, respectively, and incubated for 24 h at either 4° C. or 37° C., respectively.

(iii) In vitro assay of peptide stability—Peptides were prepared as a 1 mM solution in PBS. 20 μl of the peptide solution was diluted in 10% rat brain homogenate (in PBS+0.5% Triton X-100). The solution was incubated at 37° C. for different times, and the reaction was stopped by adding the Complete mixture of protease inhibitors (Roche Molecular Biochemicals, Mannheim, Germany). For ANA5 and 9M S.A., the bulk of the brain proteins (but not the peptides) were precipitated in cold methanol (1:4 (v/v) mixture/methanol) for 1 h at −20° C. The precipitated proteins were pelleted by centrifugation (10.000 g, 10 min, 4° C.). The supernatant containing the peptide was concentrated five times under vacuum and separated by reversed-phase HPLC (RP-HPLC). Due to recovery issues with the longer ANA1 and RI-ANA1 peptides, samples were instead lyophilized, extracted in TFA, centrifuged to remove insoluble material and separated by RP-HPLC. The area of the peak (UV absorbance at 205 nm) corresponding to the intact peptide was measured and compared with an equivalent sample incubated in PBS.

(iv) Thioflavin T assays—This method was adapted from Cell, E. et al (2011). Briefly, Aβ42 oligomers were centrifuged (21,000 g, 4° C., 1 min) to pellet insoluble material. The clarified supernatant (4.5 μg/10 μL of 100 μM stock) was added to a black-walled, clear bottom 96 well microplate (Perkin-Elmer) in triplicate. 200 uL of Thioflavin T (ThT) (5 μM in 50 mM Glycine NaOH; pH 8.5, 0.22 μM filtered) was added and the plates were read at 3-5 minutes post-addition in a FLUOSTAR OPTIMA instrument (excitation filter: 450 nm; emission filter: 490 nm; 30 s mix before reading; gain-adjust to highest reading). Samples were assayed in triplicate and the blank ThT fluorescence was subtracted from all readings. Candidate peptides were also assayed in the absence of Aβ42 for interference in the assay.

(v) Cell culture, treatment and neurotoxicity assay—M17 neuroblastoma cells were cultured similar to Taddei, K. et al (2010). Briefly, M17 cells were seeded in a 48 well plate (25000 cells/well in 500 μl 1:1 DMEM/Nutrient Mixture F12 (DMEM/F12) (Life Technologies)+10% fetal calf serum) overnight. 100 μM Aβ42 oligomer stocks (+/−candidate peptides) were diluted to 20 μM concentration in treatment medium (20% (v/v) Ham's F12, 80% (v/v) DMEM (no phenol red)) and used to treat cells (50-60% confluence) for 4 days (37° C., 5% CO2). On the fourth day, the evaluation of neurotoxicity was performed by measuring release of lactate dehydrogenase (LDH) (CytoTox-ONE™ Homogeneous Membrane Integrity Assay, Promega) and cell viability (CellTiter960Aqueous One Solution Cell Proliferation Assay (MTS), Prornega), according to the manufacturer's recommendations. Samples were assayed in triplicate.

(vi) SDS-PAGE and Western Immunoblotting—Samples for gel analysis were prepared in 2 ways, i.e. denaturing and nondenaturing conditions. Non-denatured samples were separated by electrophoresis using a modified Blue Native PAGE (BN-PAGE) protocol, as described in Miles, L. A. et al (2008). Samples were diluted in 3-(N-morpholino)propanesulfonic acid (MOPS) loading buffer (50 mM MOPS, 50 mM Tris, 20% Glycerol, 0.05% Coomassie, pH 7.7) without reducing agent and were not-heat denatured prior to PAGE separation. Where insoluble material was present in the samples, denaturing conditions were employed to solubilise this material. In the denaturing conditions, samples were diluted in SDS-PAGE loading buffer plus reducing agent (NuPAGE®), and were heat-denatured (72° C., 10 min) prior to separation of proteins by SDS-PAGE using NuPAGE® Novex 4-12% Bis-Tris gradient gels (Life Technologies). Samples were transferred onto either nitrocellulose (denatured samples) or PVDF (non-denatured samples) membranes using the iBlot® blotting system (Life Technologies). Samples were subjected to Western Immunoblotting with WO2 antibody (kindly provided by Prof. Colin Masters, University of Melbourne, Australia), to detect A species. Bands were visualised using enhanced chemiluminescence detection and exposure to X-ray film.

(vii) Atomic force microscopy (AFM)—Samples for AFM were prepared and analysed according to Stine. W. B. et al (2011). Briefly, Aβ samples were spotted on the freshly cleaved grade V1 muscovite mica and incubated for 5 minutes. Samples were then rinsed with 0.22 μm syringe-filtered double-distilled water and blow dried with several gentle pulses of compressed air (N2). Samples were then visualized under the AFM (NT-MDT) using semi-contact mode with the following parameters: a minimum contact force, amplitude between 0.5-2V (depending on the cantilever) to generate magnitude ˜20 nA, and scan rates ˜0.5-1 Hz. All data were processed using Nova NT-MDT Software v1.1.0.1780.

(viii) Surface Plasmon Resonance/Biacore assays—These experiments were performed using a Biacore 3000 Instrument (GE Healthcare) with a protocol modified from Taylor, M. et al (2010). Either 15M S.A. peptide, or Aβ42 monomer/aggregates, were immobilised on separate CM5 sensorchips (GE Healthcare). RI-ANA1 was immobilised on a CM5 sensorchip via amine coupling, according to the manufacturer's instructions, to a level of 245 resonance units (RU) (1 RU=1 pg of protein/mm2). Monomeric, oligomeric and fibrillar Aβ42 preparations were immobilised on another CM5 sensorchip (10 μM in 10 mM sodium acetate buffer (pH 4.0), injection for 5 min at a flow rate of 30 μL/min) to final levels of 9084RU, 11467RU and 4924 RU, respectively. Note that the oligomeric Aβ42 and fibrillar Aβ42 preparations were prepared 24 h prior to immobilisation, whereas the monomeric Aβ42 was prepared immediately prior to immobilisation to minimise its aggregation. For both sensorchips, a reference surface was prepared in parallel (with no addition of peptide), and used for subtraction of non-specific binding. Sensorgrams were obtained using standard conditions of 30 μL/min flow rate and HBS-EP running buffer (0.01M HEPES pH 7.4; 0.15M NaCl, 3 mM EDTA, 0.005% v/v Surfactant P20, (GE Healthcare)) as outlined in the text.

(ix) Coimmunoprecipitation analysis—Alexa₄₈₈-labeled oAβ42 was prepared as described in Jungbauer, L. M. et al (2009) and incubated with TMR-labelled RI-ANA1 in 300 μl reactions in TBS+0.05% Tween-20 (TBST) with gentle rotation for 16 hours at 4° C. Complexes were captured using 1 μg of 6E10 antibody (Covance) for Aβ (2 h, 4° C.) and then gamma-bind sepharose/Protein G-Mag Sepharose (GE Healthcare) (2 h, 4° C.). Following removal of the supernatant, the beads were washed (3×500 μl TBST) and samples were separated by denaturing SOS-PAGE on 4-12% Bis-Tris NuPAGE gels (Life Technologies). Fluorescently-labelled peptides were visualised by in gel fluorescence using a Typhoon FLA 9000 imaging system (GE Healthcare).

(x) Immunohistochemistry—Brains were obtained from 8 month old 5× FAD AD model mice Oakley, F-F et al (2006) or age-matched non-transgenic controls and post-fixed and embedded in the freezing medium as described in Drummond, E. S. et al (2013). Serial sagittal cryosections were cut at a thickness of 10 μm. Positive control staining for amyloid was performed using Thioflavin S (ThioS) as described in Youmans, K. L, et al (2012). After the removal of the freezing medium by serial changes of TBS pH 7.4, the slide was incubated for 1 h at room temperature in blocking buffer (10% goat serum in TBS). The slides were then stained with 1 μM TMR-labelled 15M S.A. peptide in 2% goat-serum-PBS for 2 hours at room temperature. Excess unbound peptide was removed prior to mounting by extensive washing with TBS. The coverslip was mounted using Prolong Gold mounting media (Life Technologies).

(xi) Brain uptake of 3H-labelled RI-ANA1 following intravenous administration—Animal experiments were approved by the Monash Institute of Pharmaceutical Sciences Animal Ethics Committee and were performed in accordance with the Australian National Health and Medical Research Council (NHMRC) guidelines for the care and use of animals for scientific purposes. Male Swiss Outbred mice (6-8 weeks of age; 25-30 g) were used in the studies and had free access to food and water during the experimental periods. An aliquot (50 μl) containing 10 μCi of 3H-labelled RI-ANA1 was administered to mice by tail vein (i.v.) injection. Brain and plasma samples were collected over a 0.5-4 h period and the concentration of RI-ANA1 in plasma and brain homogenate was performed by liquid scintillation counting (Tri-Garb 2800 TR; PerkinElmer, Boston, Mass.). The brain concentrations were corrected by subtracting the brain microvascular volume (0.035 mL/g) using 14C-sucrose as a vascular marker Bitan, G. et al (2005). As described previously, Funke, S. A. at al (2012), the brain to plasma ratio of ³H-labelled RI-ANA1 was determined using the following formula: (corrected number of disintegrations per minute [dpm] per gram of brain tissue)/(number of dpm per milliliter of plasma). The intactness of ³H-RI-ANA1 at post-dose time points following intravenous administration was assessed by HPLC. Briefly, brain samples were homogenized in a volume of MilliQ water (in mL) equal to twice the weight (in g) of the tissue using glass rod. To 300 μL. brain homogenate or 100 μL of plasma, the same volume of acetonitrile (ACN) was added prior to centrifugation. An aliquot (100 μl) of the supernatant was then loaded onto a Waters Symmetry C18 column (5×4.6 mm). Mobile phase A consisted of 0.1% v/v TFA in MilliQ water and mobile phase B consisted of 60% v/v ACN in 0.1% v/v TEA in MilliQ water, ³H-15M S.A. peptide was analysed using the following gradient profile: 0 min, 95% A; 0-10 min, 70% A; 10-12 min, 95% A. The eluant from the column was collected every 0.5 min and the dpm of each fraction was measured by liquid scintillation counting. The profiles (dpm vs time) of brain and plasma samples were then generated and compared with those of ³H-RI-ANA1 solution as control.

### EXAMPLE 6A

### In Vitro Stability of Analogue Peptides with Increased Therapeutic Potential

The in vitro stability of ANA1 (“15 mer”), ANA5 (“9 mer”), 15M S.A. (RI-ANA1), 9M S.A., was measured following incubation in dilute rat brain homogenate and HPLC quantification.

Peptides were prepared as 1 mM solutions in PBS and diluted in 10% rat brain homogenate. Solutions were incubated at 37° C. for different times, and the reactions were stopped by addition of protease inhibitors. Samples were processed as described herein in the General Materials/Methods, separated by RP-HPLC and the level of intact peptide was determined at each time point.

We found that trace amounts of unmodified 15 mer and none of the unmodified 9 mer were present at t=30 min (Table 5). In contrast, the concentrations of both stable analogue peptides remained relatively unchanged following an extended incubation of 300 min (Table 5).

TABLE 5

| Time | 15mer | 15M S.A. | 9mer | 9M S.A. |
|---|---|---|---|---|
| (min) | (μg) | (μg) | (μg) | (μg) |
| 0 | 25.78 | 29.80 | 12.00 | 14.11 |
| 15 | 5.55 | 27.80 | 0.47 | 12.35 |
| 30 | 0.03 | 29.40 | 0 | 12.35 |
| 300 | 0.02 | 33.50 | 0 | 12.51 |

### EXAMPLE 6B

### Effects on Aβ42 Aggregation and Neurotoxicity

Given the neurotoxicity attributed to oligomeric Aβ42, we assessed the ability of the stable analogues to influence their formation and toxicity. We incubated monomeric Aβ42 peptide under conditions specifically favouring oligomerisation for 24 h, Stine, W. B. et al (2011), in the presence or absence of the candidate peptides.

At t=24 h, soluble and insoluble fractions (where present) were assayed for the presence of Aβ42 by SDS-PAGE and Western blotting. We then assayed the Aβ42 aggregation in these samples using ThT fluorescence assays and BN-PAGE/Western blotting. We additionally diluted samples to 20 μM Aβ42 concentration, treated M17 neuroblastoma cells and quantitated neurotoxicity following 4 days of treatment.

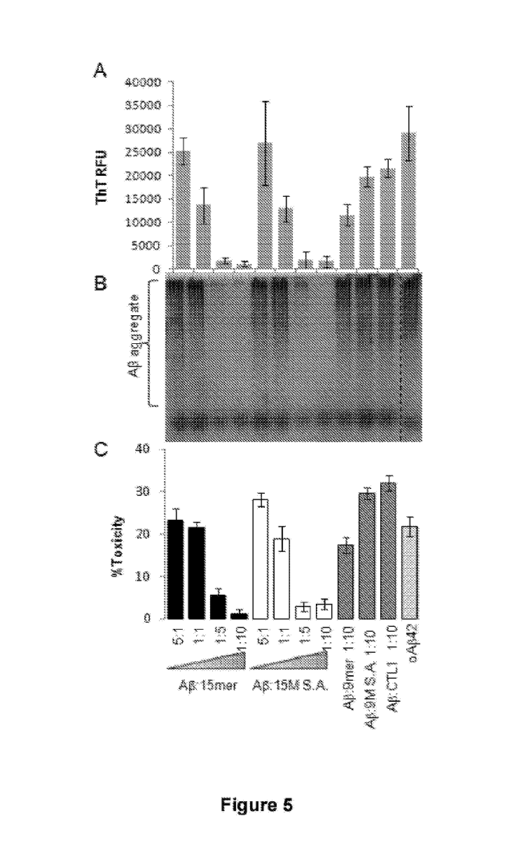

ThT analysis revealed that both the 15 mer and 15M S.A. peptide reduced ThT fluorescence (and thus Aβ42 aggregation and oligomerisation) in a dose-dependent manner and with similar potency (FIG. 5A). In comparison, the 9 mer peptide showed similar activity, but reduced potency, and the 9M S.A. peptide showed overlapping activity with a scrambled control peptide (CTL1) (FIG. 5A). These findings were mimicked by the BN-PAGE and Western Blotting analysis (FIG. 5B), where the presence of 15 mer and 15M S.A. resulted in a dose-dependent reduction in the Aβ42 aggregation “smear”. The LDH assay indicated that a reduction in Aβ42 aggregation correlated with reduced neurotoxicity, with the 15 mer and 15M S.A. reducing neurotoxicity in a dose-dependent manner and with similar potency (FIG. 5C). In contrast, the 9 mer offered less potent neuroprotection and the activity of the 9M S.A. peptide was again similar to the scrambled control peptide (FIG. 5C). The neuroprotection offered by the peptides was also confirmed using MTS assays, which indicated increases in cell viability.

### EXAMPLE 6C

### Effects on Formation of Non-Toxic, Insoluble Aggregates During Aβ42 Oligomerisation

The ThT and Western Blotting analysis described above indicated that the 15M S.A. peptide reduced the formation of soluble Aβ42 oligomers (FIGS. 5A and 5B). We used denaturing SDS-PAGE and Western Blotting analysis to assess the relative amounts of soluble versus insoluble Aβ42 species.

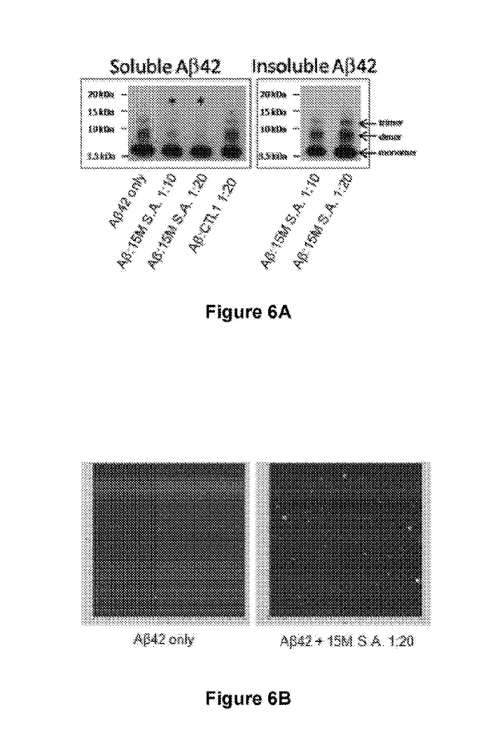

As in FIG. 5B, we found that the presence of 15M S.A. resulted in a dose-dependent reduction in soluble Aβ42 aggregates (FIG. 6A, left panel), whereas the control peptide resulted in similar soluble Aβ42 aggregates as the Aβ42 only sample (FIG. 6A, left panel). Increasing concentrations of 15M S.A. peptide resulted in increasing amounts of insoluble material, whereas no insoluble deposits were seen for the Aβ42 only sample, or in the presence of the control peptide. Western Blotting revealed that Aβ42 aggregates were indeed present in the insoluble deposits, and the dose-dependent reduction in soluble Aβ42 aggregates in the presence of 15M S.A. was concurrent with an increase in insoluble Aβ42 aggregates (FIG. 6A, right panel).

Using atomic force microscopy (AFM), we found that Aβ42 oligomers formed in the absence of 15M S.A. were predominantly 1.5-4 nm diameter, with a few larger aggregates of 8-12 nm diameter (FIG. 6B, left panel). However, Aβ assemblies formed in the presence of 15M S.A. ranged from smaller (2-6 nm diameter) to extreme (25-30 nm) size (FIG. 6B, right panel). Taken together, it appeared that the 15M S.A. peptide bound Aβ42, altered its oligomerisation and promoted the formation of non-toxic aggregates.

### EXAMPLE 6D

### Binding to Pre-Formed Aβ42 Oligomers

Using two complementary measures of protein-protein interactions; Surface Plasmon Resonance/Biacore assays and coimmunoprecipitation analysis we assessed whether 15M S.A. could also interact with pre-formed Aβ42 oligomers (oAβ42). For these assays, we utilised two stable analogue control peptides (CTL1 S.A. and CTL2 S.A.), which were validated in ThT and LDH assays and found to mimic the activity of the CTL1 peptide used in previous assays.

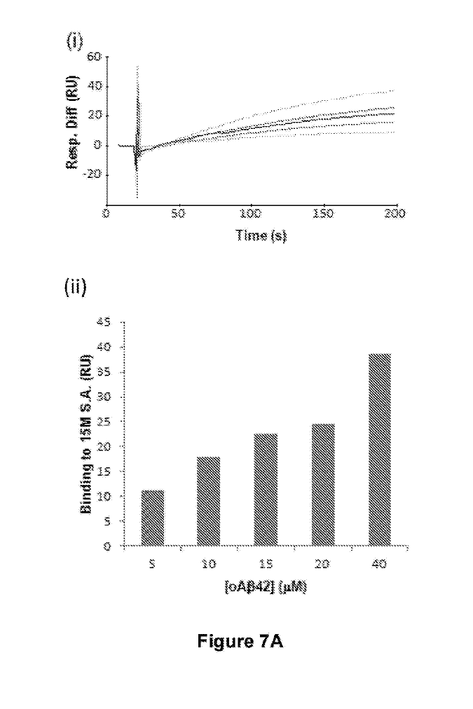

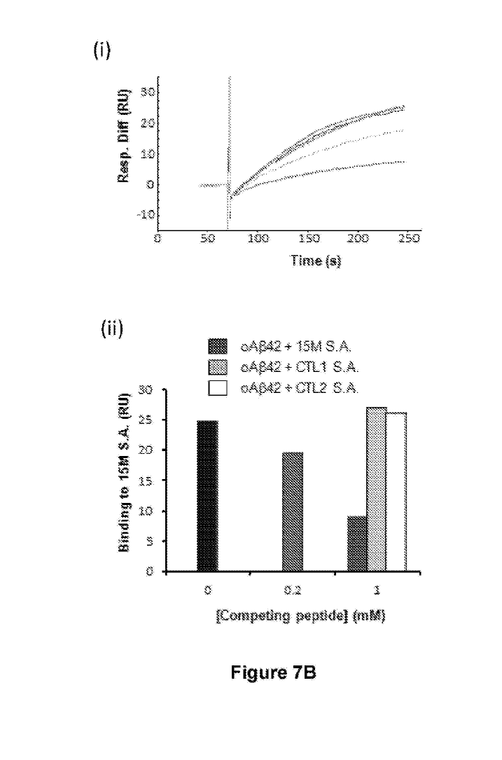

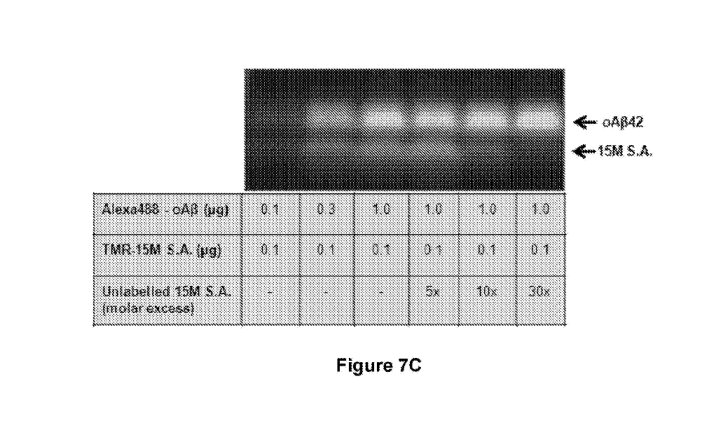

For Surface Plasmon Resonance assays, 15M S.A. was immobilised on a Biacore sensorchip and oAβ42 concentrations over 5-40 μM were injected and monitored for binding to 15M S.A. It was evident that oAβ42 bound 15M S.A in a dose-dependent manner (FIG. 7A).

To confirm the specificity of this interaction, we performed solution competition assays where free 15M S.A. peptide or control peptides were co-injected with oAβ42. For these studies, we used an oAβ42 concentration of 20 μM, which was found to give reproducible responses in replicate injections over multiple cycles in a given experiment. We found that increasing concentrations of free 15M S.A. in solution resulted in a dose-dependent reduction in oAβ42 binding the immobilised 15M S.A. on the sensorchip (FIG. 7B). However, equivalent concentrations of two different control peptides could not mimic this action and did not reduce the amount of oAβ42 binding to immobilised 15M S.A. (FIG. 7B).

In order to confirm these findings, we performed complementary studies involving coimmunoprecipitation analysis. Here, Alexa488-labelled oAβ42 was incubated with TMR-labelled 15M S.A. peptide to promote the formation of a protein complex, which was captured by immunoprecipitation using 6E10 antibody to bind oAβ species. Denaturing SDS-PAGE was used to separate the protein complexes, and oAβ42 and 15M S.A. were visualised via their respective fluorescent labels. The 15M S.A. peptide coimmunoprecipitated with oAβ42 in a dose-dependent manner, (FIG. 7C). Furthermore, the amount of labelled, coimmunoprecipitated 15M S.A. peptide could be reduced by competition in solution with increasing quantities of unlabelled 15M S.A. peptide (FIG. 7C). These findings confirmed the results of the Surface Plasmon Resonance assays and collectively indicated that the 15M S.A. peptide directly interacted with oAβ42.

We extended the Surface Plasmon Resonance analysis to obtain an estimate of the affinity of the interaction between 15M S.A. and oAβ42, by fitting the experimental data to models within the BiaEvaluation™ v4.1 software. It is crucial to highlight that this value is only an overall estimate of binding affinity across the entire spectrum of oligomers present in the oAβ42 preparation, which is a non-homogeneous mixture of aggregates. The best fit occurred for a 1:1 binding model with drifting baseline correction (Chi²=0.214) and yielded a KD value of low micromolar value (11 μM), indicating a moderate affinity between the 15M S.A. peptide and oAβ42.

### EXAMPLE 6E

### Binding to Monomeric, Oligomeric and Fibrillar Aβ42

We further investigated the ability of 15M S.A. to bind less aggregated Aβ preparations (monomeric (m) Aβ42) and more aggregated preparations (fibrillar (f) Aβ42). To allow a comparison of 15M S.A. binding to different Aβ species, a sensorchip was generated with immobilised monomeric, oligomeric and fibrillar Aβ42 preparations as described in the General Materials/Methods. A series of 15M S.A. concentrations were injected across the surfaces, monitored for binding to the respective Aβ42 species, and the data was corrected for the relative amount of immobilised material on the individual flow cells.

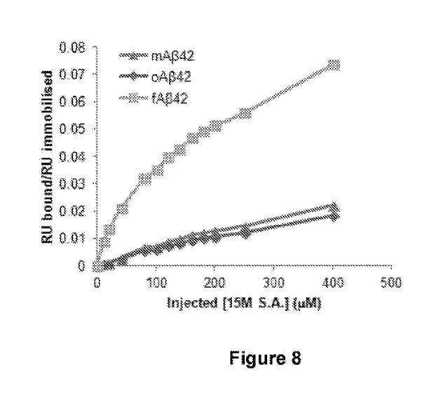

We found that 15M S.A. interacted with all of the Aβ42 species in a concentration-dependent manner, but the highest magnitude of binding was seen for the Aβ42 fibrils. (FIG. 8).

### EXAMPLE 6F

### Detection of Amyloid Plaques

We investigated whether TMR-labelled 15M S.A. could be used to stain amyloid plaques ex vivo using brain tissue sections from AD model mice, in comparison with Thioflavin S (Thio S), which binds mature amyloid deposits and readily detects these plaques.

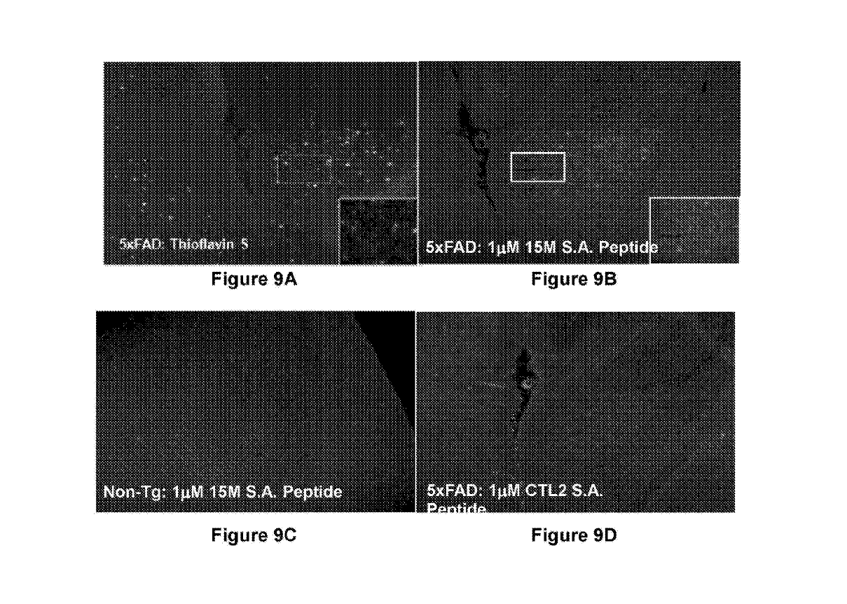

In ex vivo staining of brain tissue from 8 month old 5× FAD AD model mice, Thio S staining revealed extensive plaques within the brain (FIG. 9A). In comparison, serial sections treated with the TMR-labelled 15M S.A. peptide also resulted in staining of some amyloid deposits, but to a lesser extent than Thio S. (FIG. 9B). Notably, the TMR-labelled 15M S.A. peptide did not stain control (non-AD) brain tissue from age-matched control mice in related experiments (FIG. 9C). Additionally, a TMR-labelled control peptide did not result in any comparable staining of amyloid deposits in the 5× FAD brain tissue (FIG. 9D).

### EXAMPLE 6G

### Crossing the Blood Brain Barrier (BBB)

In order to assess the ability of the 15M SA peptide to cross the BBB in vivo, 10 μCi of tritiated peptide was administered by i.v. injection to mice as described in the General Materials/Methods, and its concentration in brain and plasma was determined in samples collected over a 0.5-4 h period.

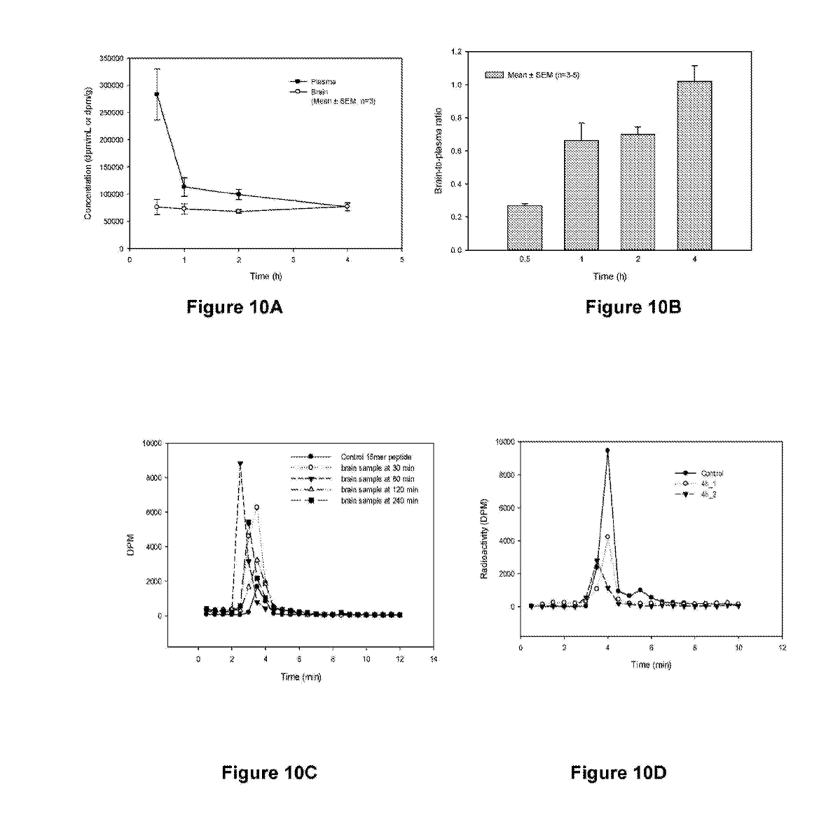

Scintillation counting revealed that the plasma concentrations of tritiated peptide dropped over time, as in a normal i.v, profile. However, the tritiated peptide was detected in brain at the initial t=30 min time point and remained relatively constant until t=4 h, indicating an accumulation with time (FIG. 10A) and presenting as an increasing brain to plasma ratio over time (FIG. 10B).

We further performed control experiments to ensure that the measured radioactivity in brain and plasma samples corresponded to intact ³H-15M S.A peptide, rather than free label or degradation products (FIG. 10C, D). There was no apparent shift in the retention time of ³H-labelled 15M S.A.in brain and plasma samples collected at designated time points, suggesting that the majority of detected radioactivity was due to intact peptide.

### REFERENCES

Sullivan P M, Mezdour H, Aratani Y, Knouff C, Najib J, Reddick R L, Quarfordt S H, Maeda N. Targeted replacement of the mouse apolipoprotein E gene with the common human APOE3 allele enhances diet-induced hypercholesterolemia and atherosclerosis. J Biol Chem 1997; 272:17972-17980.

Sharman M J, Morici M, Hone E, Berger T, Taddei K, Martins I J, Lim W L, Singh S. Wenk M R, Ghiso J, Buxman J D, Gandy S, Martins R N. APOE genotype results in differential effects on the peripheral clearance of amyloid—beta 42 in APOE knock-in and knock-out mice. J Alzheimers Dis. 2010; 21:403-409.

Taddei, K., Laws, S. M., Verdile, G., Munns, S., D'Costa, K., Harvey, A. R., Martins, I. J., Hill, F., Levy, E., Shaw, J. E., and Martins, R. N. (2010) Novel phage peptides attenuate beta amyloid-42 catalysed hydrogen peroxide production and associated neurotoxicity. Neurobiol Aging 31, 203-214

Stine, W. B., Jungbauer, L., Yu, C., and LaDu, M. J. (2011) Preparing synthetic Abets in different aggregation states. Methods Mol Biol 670, 13-32

Cerf, E., Gustot, A., Goormaghtigh, E., Ruysschaert, J. M., and Raussens, V. (2011) High ability of apolipoprotein E4 to stabilize amyloid-beta peptide oligomers, the pathological entities responsible for Alzheimer's disease. FASEB J 25, 1585-1595

Miles, L. A., Wun, K. S. Crespi, G. A., Fodero-Tavoletti, M. T., Galatis, D., Bagley, C. J., Beyreuther, K, Masters, C. L., Cappai, R., McKinstry, W. J., Barnham, K J., and Parker, M. W. (2008) Amyloid-beta-anti-amyloid-beta complex structure reveals an extended conformation in the immunodominant B-cell epitope. J Mol Biol 377, 181-192

Taylor, M., Moore, S., Mayes, J., Parkin, E., Beeg, M., Canovi, M., Gobbi, M., Mann, D. M , and Allsop, D (2010) Development of a proteolytically stable retro-inverso peptide inhibitor of beta-amyloid oligomerization as a potential novel treatment for Alzheimer's disease, Biochemistry 49, 3261-3272

Jungbauer, L. M., Yu, C., Laxton, K. J., and LaDu, M. J. (2009) Preparation of fluorescently-labeled amyloid-beta peptide assemblies: the effect of fluorophore conjugation on structure and function. J Mol Recognit 22, 403-413

Oakley, H., Cole, S. L., Logan, S., Maus, E., Shao, P., Craft, J., Guillozet-Bongaarts, A., Ohno, M., Disterhoft, J., Van Eldik, L., Berry, R. and Vassar, R. (2006) Intraneuronal beta-amyloid aggregates, neurodegeneration, and neuron loss in transgenic mice with five familial Alzheimer's disease mutations: potential factors in amyloid plaque formation. J Neurosci 26, 10129-10140

Drummond, E. S., Muhling, J., Martins, R. N., Wijaya, L. K., Ehlert, E. M., and Harvey, A. R. (2013) Pathology associated with AAV mediated expression of beta amyloid or C100 in adult mouse hippocampus and cerebellum. PLoS One 8, e59166

Youmans, K. L., Tai, L. M., Nwabuisi-Heath, E., Jungbauer, L., Kanekiyo, T., Gan, M., Kim, J., Eimer, W. A., Estus, S., Rebeck, G. W., Weeber, E. J., Bu, G., Yu, C., and Ladu, M. J. (2012) APOE4-specific changes in Abeta accumulation in a new transgenic mouse model of Alzheimer disease. J Biol Chem 287, 41774-41786

Bitan, G., Fradinger, E. A., Spring, S. M., and Teplow, D. B. (2005) Neurotoxic protein oligomers—what you see is not always what you get. Amyloid 12, 88-95

Funke, S. A., Bartnik, D., Gluck, J. M., Piorkowska, K., Wiesehan, K., Weber, U., Gulyas, B., Hakim, C., Pfeifer, A., Spenger, C., Mulls, A., and Willbold, D. (2012) Development of a small D-enantiomeric Alzheimer's amyloid-beta binding peptide ligand for future in vivo imaging applications. PLoS One 7, e41457

1-18. (canceled)

19. An Aβ modulating peptide comprising the amino acid sequence Arg-Lys-Leu-Met-Gln-Pro-Thr-Arg-Asn-Arg-Arg-Asn-Pro-Asn-Thr (SEQ ID NO:2) wherein all of the amino acids are D-amino acids.

20. An Aβ modulating peptide according to claim 19 further comprising a detectable label.

21. An Aβ modulating peptide according to claim 19 further comprising a water soluble polymer.

22. An Aβ modulating peptide according to claim 20 further comprising a water soluble polymer.

23. A pharmaceutical composition comprising an Aβ modulating peptide according to claim 19 and a pharmaceutically acceptable carrier.

24. A method for modulating Aβ aggregation, Aβ neurotoxicity and/or Aβ peripheral clearance comprising the step of contacting Aβ with an Aβ modulating peptide according to claim 19 or a pharmaceutical preparation comprising the Aβ modulating peptide and a pharmaceutically acceptable carrier.

25. A method for treating a subject suffering from amyloidosis comprising the step of administering to said subject an effective amount of an Aβ modulating peptide according to claim 19 or a pharmaceutical preparation comprising the Aβ modulating peptide and a pharmaceutically acceptable carrier.

26. A method of treating a subject to slow or stop the progression of amyloidosis comprising the step of administering to said subject an effective amount of an Aβ modulating peptide according to claim 19 or a pharmaceutical preparation comprising the Aβ modulating peptide and a pharmaceutically acceptable carrier.

27. A method according to claim 26 wherein the progression is between at least two disease stages selected from (i) preclinical amyloidosis (ii) mild cognitive impairment (MCI) and (iii) amyloid mediated dementia, such as Alzheimer's disease.

28. A method according to claim 25 wherein the amyloidosis is Alzheimer's disease.

29. Use of an Aβ modulating peptide according to claim 19 or a pharmaceutical preparation comprising the Aβ modulating peptide and a pharmaceutically acceptable carrier for detecting Aβ.

30. Use according to claim 29 wherein the detection is in vivo.

31. Use according to claim 29 wherein the detection is in vitro.

32. Use according to claim 29 for diagnosing amyloidosis in a subject.

33. A method for detecting Aβ comprising the step of contacting a sample with an Aβ modulating peptide according to claim 19 or a pharmaceutical preparation comprising the Aβ modulating peptide and a pharmaceutically acceptable carrier and detecting the formation of a complex between the Aβ and the Aβ modulating peptide.
